(19) United States
(12) Patent Application Publication
(10) Pub. No.: US 2018/0117176 A1
(43) Pub. Date: May 3, 2018

(54) POROUS SILICON MICROPARTICLE-BASED CANCER VACCINES AND METHODS FOR POTENTIATING ANTI-TUMOR IMMUNITY

(71) Applicant: The Methodist Hospital, Houston, TX (US)

(72) Inventors: Haifa SHEN, Bellaire, TX (US); Xiaojun XIA, Pearland, TX (US); Mauro FERRARI, Houston, TX (US)

(21) Appl. No.: 15/832,771

(22) Filed: Dec. 6, 2017

Related U.S. Application Data
(63) Continuation of application No. PCT/US2016/025757, filed on Apr. 1, 2016.
(60) Provisional application No. 62/142,278, filed on Apr. 2, 2015.

(51) Int. Cl. A61K 47/69; A61K 39/39; A61K 39/00; A61K 39/12; B82Y 5/00; C12N 5/0783; A61K 9/51; C12N 15/113; A61P 35/04

(52) U.S. Cl. CPC A61K 47/6923; A61K 39/39; A61K 39/0011; A61K 39/12; B82Y 5/00; A61K 47/6929; C12N 2770/24134; A61K 9/5115; C12N 15/1138; A61P 35/04; A61K 2039/5154; A61K 2039/55555; A61K 2039/57; C12N 5/0637

(57) ABSTRACT

Porous silicon (pSi) microparticles (PSM) are disclosed, which provide an important advance in the area of cancer immunotherapeutics and molecular nanomedicine. In particular, potent PSM-based adjuvants are disclosed for dendritic cell-based vaccines compositions, and methods for their use in a variety of cancer immunotherapies. The PSM of the present invention are also useful in developing other types of vaccines, including those not necessarily related to the treatment of cancers, such as vaccines for the treatment of acne, Alzheimer's disease, asthma, atherosclerosis, autoimmune disorders, autoinflammatory disease, celiac disease, colitis, Crohn's disease, diabetes, glomerulonephritis, infectious diseases, inflammatory bowel disease, irritable bowel syndrome, ischemia, Lupus, pelvic inflammatory disease, reperfusion injury, rheumatoid arthritis, sarcoidosis, transplant rejection, West Nile virus, and related illnesses.

### CROSS-REFERENCE TO RELATED APPLICATIONS

The present application is a continuation of PCT International Patent Application No. PCT/US2016/025757, filed Apr. 1, 2016 (pending; Atty. Dkt. No. 37182.190WO01), which claims priority to United States Provisional Patent Application No. 62/142,278, filed Apr. 2, 2015 (expired; Atty. Dkt. No. 37182.190PV01); the contents of each of which is specifically incorporated herein in their entirety by express reference thereto.

### STATEMENT REGARDING FEDERALLY SPONSORED RESEARCH DEVELOPMENT

This invention was made with government support under Grant Nos. W81XWH-09-1-0212 and W81XWH-12-1-0414 awarded by the United States Department of Defense; and Grant Nos. U54-CA143837 and U54-CA151668 awarded by the National Institute of Health. The government has certain rights in the invention.

### BACKGROUND OF THE INVENTION

### NAMES OF THE PARTIES TO A JOINT RESEARCH AGREEMENT

Not Applicable.

### Field of the Invention

The present disclosure relates to the fields of molecular biology, oncology and nanomedicine. Porous silicon microparticles have been developed for use in a variety of therapeutic indications. In particular embodiments, these compositions function as potent adjuvants to facilitate the development of cancer vaccines. Additionally, methods and medicaments are also provided for enhancing antigen cross-presentation, inducing mammalian type I interferon-specific cellular responses, and treating various cancers and tumors in affected mammals.

### Description of Related Art

Cancer immunotherapy seeks to boost or restore immune function for effective recognition of antigens associated with aberrant cells. The extent of such immunotherapy-based approaches is broad, and includes cytokine delivery to stimulate a passive immune response (Wayteck, et al., 2013; Jaime-Ramirez et al., 2011), ex vivo stimulation of autologous immune cells that are subsequently administered to a patient (Phan et al., 2013), the use of toxic chemotherapy adjuvants for stimulating an immune response (Wan et al., 2012), antibody-mediated therapy (Lan et al., 2013), and formulations consisting of antigens combined with alum, emulsions, liposomes, immune stimulating complexes (Audibert, 2003), or polymeric nanoparticles (Craparo and Bondi, 2012).

The vaccine adjuvant alum, thought to function as a depot for sustained antigen release, induces cytotoxic effects leading to the release of uric acid and recruitment of immune cells to the site of injection (Kool et al., 2008). Alum favors T helper 2 (Th2) immune responses, which induce B cells to produce neutralizing antibodies (Rimaniol et al., 2007; Mori et al., 2012; Brewer, 2006). However, effective cancer immunotherapies require Th1 cytokines to arrest tumor growth, specifically IFN-γ and TNF-α (Braumuller et al., 2013).

Similar to alum, cationic liposomes have inherent cytotoxicity, inducing cell death and stimulating immune cell infiltration to the site of injection or accumulation. In contrast to alum, which relies on surface absorption for binding of MPL, liposomes incorporate MPL into the lipid bilayer. Previously, it was reported that MPL-liposomes suppress tumor growth in a 4T1 immune competent murine model of breast cancer, unlike an equivalent dose of free MPL (Meraz et al., 2014). In addition to recruiting and activating immune cells, tumor cell damage caused by the cationic liposomes is proposed to release endogenous tumor antigens, directing the immune response against cancer cells.

The large pool of endogenous tumor antigens creates an array of epitopes for immune recognition. The adjuvant effects of cationic liposomes were demonstrated by Yan et al. (2007), who showed by microarray mRNA analysis that DOTAP liposomes up-regulated chemokines (including CCL2, CCL3, and CCL4) in dendritic cells (DC). Incorporation of cholesterol in the bilayer of cationic liposomes also enhanced their adjuvant effect (Barnier-Quer et al., 2013). Using porous silicon microparticles, it was previously shown that particle presentation of adsorbed MPL increases particle uptake by DC; elevated DC expression of co-stimulatory and major histocompatibility complex (MHC) class I and II molecules; increased migration of DC to the draining lymph node; and enhanced associations between DCs presenting an ovalbumin peptide sequence, and T cells from OT-1 mice (Meraz et al., 2012).

Aluminum-based adjuvants, designed to enhance immune responses to antigens, were introduced in the 1920s, and 91 years later, they remain the standard adjuvant used in vaccines. Studies on new nanoparticle-based vaccines are increasing, with the advantage of combining adjuvant and antigen in a single entity. These particles were rapidly internalized by antigen-presenting cells (APC) with trafficking to lymphoid tissue for presentation to lymphocytes.

### BRIEF SUMMARY OF THE INVENTION

The present invention overcomes these and other inherent limitations in the art by providing, in a general sense, mesoporous silicon particles for use in a variety of diagnostic and therapeutic indications. In illustrative embodiments, pSi microparticles for use as adjuvants and as vaccines for anti-cancer immunity have been described.

In particular embodiments, the present disclosure provides porous silicon (pSi) microparticles (PSM) as potent adjuvants for cell-based (and in particular, mammalian dendritic cell-based) vaccines. Such adjuvants find particular use in the preparation of medicaments such as vaccines for treatment of one or more diseases, disorders, deficiencies, dysfunctions, or defects in a mammal, and particularly for the treatment and/or amelioration of one or more symptoms of a mammalian cancer, such as a human tumor. The PSM disclosed herein find particular utility in a variety of in vitro, ex vivo, and in vivo treatment regimens, and they may be formulated alone, or, alternatively, in combination with one or more additional agents, including, without limitation, one or more tumor antigen(s), one or more antigenic peptides, one or more diagnostic reagents, one or more therapeutic reagents, one or more cytotoxic reagents, one or more chemotherapy agents, or any combination thereof, for use in a variety of therapeutic indications, including, without limitation, for the treatment or amelioration of symptoms of one or more human cancers or other hyperproliferative disorders.

The PSM disclosed herein, may be prepared in accordance with one or more of the methods described in the examples that follow, and in an illustrative embodiment, may be incubated with patient-derived dendritic cells in a clinical laboratory, such that those cells are then subsequently injected back into the patient's body, wherein they stimulate, or otherwise elicit the production of cytotoxic T-cells within the patient's body, thereby facilitating a process for killing tumor cells in situ.

In an analogous fashion, PSM may also be packaged ex situ with one or more antigens that are specific to the particular targeted disease, and then incubated with patient-derived dendritic cells, which are subsequently injected back into the patient to stimulate or otherwise elicit an immune response within the patient's body, thereby aiding in the body's defense against the particular targeted disease.

Alternatively, PSM compositions can be directly injected into a patient, instead of incubation with dendritic cells in vitro, to boost immune responses.

As described herein, PSM may further optionally include one or more active agents, such as, for example, one or more prophylactic agents, one or more therapeutic agents, one or more diagnostic agents, one or more vaccines, one or more imaging agents, one or more radiolabels, one or more adjuvanting agents, one or more chemotherapeutic agents, one or more cytotoxic agents, or any combination thereof incorporated within or about the PSM, or formulated with the PSM in a pharmaceutical formulation for co-administration to a patient.

In related embodiments, the invention also provides therapeutic and/or diagnostic kits including one or more of the PSM-based adjuvant or vaccine compositions disclosed herein, typically in combination with one or more pharmaceutically acceptable carriers, one or more devices for administration of the compositions to a subject of interest, as well as one or more instruction sets for using the composition in the prevention, the diagnosis, or the treatment of a mammalian condition, disease, disorder, trauma, and/or dysfunction, including, without limitation, one or more cancers and such like.

The invention also provides in an overall and general sense, methods for providing an active agent to a mammalian dendritic cell comprising administering to the subject, an effective amount of one or more of the PSM compositions disclosed herein. In certain embodiments, the subject is at risk for, diagnosed with, or suspected of having one or more abnormal conditions, including, for example, one or more cancers or other hyperproliferative disorders.

As noted herein, the PSM-antigen loaded vaccine compositions of the present invention may further optionally include one or more additional therapeutic agents, diagnostic agents, imaging agents, or a combination thereof.

In another embodiment, the present disclosure also provides a method for administering an active agent to one or more cells, tissues, organs, or systems of a mammalian subject in need thereof. The method generally involves providing to a mammalian subject in need thereof, one or more of the compositions disclosed herein in an amount and for a time effective to administer the active agents with a population of PSM to one or more selected tissues, organs, systems, or cells within or about the body of the subject. In particularly preferred embodiments, the subject is a human, and the composition comprises a first stage particle that is adapted and configured to localize to at a first target site within or about the body of the human patient to which the active agent is being administered.

As noted herein, the compositions of the present disclosure may be administered to the subject through any one or more conventional methods for administration, including, without limitation, orally, intranasally, intravenously, subcutaneously, or by direct injection to one or more cells or one or more tissues within or about the body of the subject.

As further described herein, in certain applications, it may be desirable to contact a population of cells obtained from a subject ex vivo with the PSM compositions, and then, subsequently, to reintroduce the resulting contacted cells into the body of the subject. Such ex vivo therapy is particularly contemplated to be useful in introducing the disclosed PSM to populations of human dendritic cells, allowing the active ingredients to be contacted with the cells, and then to re-introduce the resulting cells back into the body of the animal. Preferably, the cells extracted for such ex vivo manipulation will be those of the actual patient undergoing treatment.

In particular embodiments, the PSM compositions of the present invention may be formulated for pharmaceutical administration, and preferably for administration to a human. Such compositions may further include one or more additional therapeutic agents, chemotherapeutics, adjuvants, or a second distinct population of PSM.

### BRIEF DESCRIPTION OF THE DRAWINGS

The following drawings form part of the present specification and are included to demonstrate certain aspects of the present invention. The patent or application file contains at least one drawing executed in color. Copies of this patent or patent application publication with color drawing(s) will be provided by the Office upon request and payment of the necessary fee.

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The invention may be better understood by reference to the following description taken in conjunction with the accompanying drawings, in which like reference numerals identify like elements, and in which:

FIG. 1A, FIG. 1B, FIG. 1C, FIG. 1D, FIG. 1E, FIG. 1F, and FIG. 1G show PSM-loaded antigen was efficiently internalized by dendritic cells (DCs) through phagocytosis and macropinocytosis. FIG. 1A shows representative microscopic views of DCs internalizing PSM particles loaded with ovalbumin-fluorescein isothiocyanate (OVA-FITC) nanoliposomes. Upper panel, DC2.4 cells; lower panel, bone-marrow-derived dendritic cells (BMDCs). Scale bar, 25 μm. FIG. 1B is a representative transmission electron microscopy (TEM) image showing the vesicular structure around a PSM/OVA particle inside a DC. Upper panel magnification=5,000×; lower panel magnification=50,000×. Upper panel scale bar=4 μm; lower panel bar=0.5 μm. FIG. 1C shows DC uptake of PSM/FITC-OVA in the presence of the phagocytosis inhibitor, cytochalasin D. FIG. 1D shows DC uptake of PSM/FITC-OVA in the presence of the macropinocytosis inhibitor, amiloride. FIG. lE shows IL-2 production by B3Z cells after co-incubation with DCs pre-treated for 3 hr with different concentrations of soluble OVA or PSM/OVA. FIG. IF shows IL-2 production by B3Z cells after co-incubation with DCs that were primed with 50 μg/mL soluble OVA or PSM/OVA followed by fixation at different time points. FIG. 1G shows IL-2 production by B3Z cells after co-incubation with DCs. The DCs were extensively washed after priming with 5 μg/mL soluble peptide or PSM/peptides, and either immediately co-incubated with B3Z cells (0 hrs), or cultured for 18 to 30 hrs before T-cell co-incubation; Data are presented as mean±S.D.; *, P <0.05, **, P <0.01; (See also FIG. 7A-FIG. 7E);

FIG. 2A, FIG. 2B, and FIG. 2C show antigen presentation of PSM-loaded OVA. FIG. 2A illustrates subcellular transport of antigens. DCs were incubated with PSM/FITC-OVA for 0.5 hrs, and then washed extensively to remove unbound particles. The cells were cultured for an additional 0.5 hr, 1 hr, 3 hrs, or 6 hrs before fixation, followed by immunostaining with antibodies recognizing specific organelles (EEA1 for early endosome; Rab7 for late endosome; TR for recycling endosome; and KDEL for the ER). Scale bar=25 μm. FIG. 2B shows IL-2 production by B3Z cells after co-incubation with DCs treated with 100 μg/mL OVA or PSM/OVA in the presence of the proteosome inhibitors, MG-132 and epoxomicin, or the lysosome inhibitor, leupeptin. Peptide I (100 ng/mL) served as the positive control. FIG. 2C shows OVA cross-presentation by BMDCs. BMDCs were incubated with OVA or PSM/OVA for 16 hrs, followed by harvest and labeling with the anti-CD11c antibody identifying DCs and the 25D1.16 antibody recognizing OVA₂₅₇₋₂₆₄/H-2K^{b} complex on DC surface. Percentages of 25D1.16-staining positive populations in DCs are shown in red numbers. Data are presented as mean±S.D; *, P <0.05, **, P <0.01 (see also FIG. 8A, FIG. 8B and FIG. 8C);

FIG. 3A, FIG. 3B, FIG. 3C, and FIG. 3D show PSM-induced IFN-I signaling in DCs. FIG. 3A shows protein levels of pro-inflammatory cytokines in the culture medium of BMDCs 24 hrs after incubation with PSM, free antigen, or PSM/antigen. Cell culture medium served as the negative control. FIG. 3B shows the expression pattern of co-stimulatory molecules on the surface of BMDCs 24 hrs after incubation with PSM. FIG. 3C is a QPCR analysis of mRNA levels of the Ifn-α4 and Ifn-β genes in BMDCs 5 hrs after co-incubation with PSM, free antigen, or PSM/OVA antigen. FIG. 3D shows an ELISA assay for the proteins, IFN-β and RANTES, in the cell culture medium of BMDCs 24 hrs after incubation with PSM. Data are presented as mean±S.D.; *, P <0.05, **, P <0.01 (see also FIG. 9A and FIG. 9B);

FIG. 4A, FIG. 4B, FIG. 4C, FIG. 4D, FIG. 4E, FIG. 4F, FIG. 4G, FIG. 4H, FIG. 4I, and FIG. 4J show TRIF and MAVS mediated PSM induced interferon type I (IFN-I) activation. FIG. 4A, FIG. 4B, FIG. 4C, and FIG. 4D illustrate PSM-induced changes of Ifn-α4 and Ifn-β gene expression in BMDCs infected with lentiviruses carrying shRNAs targeting the indicated genes. FIG. 4E, FIG. 4F, FIG. 4G, and FIG. 4H illustrate PSM-induced changes of Ifn-α4 and Ifn-β gene expression in BMDCs isolated from wild-type or gene knockout mice. FIG. 4I shows PSM-induced RANTES production in BMDCs isolated from wild type or gene knockout mice. FIG. 4J is a schematic view of PSM-induced signaling pathway that mediates IFN-I activation. Data are presented as mean±S.D.; *, P <0.05, **, P <0.01; (see also FIG. 10A, FIG. 10B, FIG. 10C, FIG. 10D, FIG. 10E, and FIG. 10F);

FIG. 5A, FIG. 5B, FIG. 5C, FIG. 5D, FIG. 5E, FIG. 5F, and FIG. 5G show PSM/HER2 DC vaccine inhibited mouse mammary gland tumor growth. FIG. 5A shows inhibition of primary TUBO tumor growth. TUBO cells were inoculated into the mammary gland fat pads. After 4 days, mice (n=8/group) were treated i.v. with PBS, PSM, PSM/p66, DC+p66, or DC+PSM/p66, and tumor growth was monitored in the next 4 weeks. Tumor volume is presented as mean±SEM. FIG. 5B shows tumor multiplicity in BALB-neuT mice with different treatments. The BALB-neuT transgenic mice were divided into three groups (n=5), and treated twice (weeks 6 and 8) i.v. with the indicated agents; the number of tumor nodules per mouse was counted on a weekly basis. FIG. 5C, FIG. 5D, and FIG. 5E show quantitative RT-PCR measurement on mRNA levels of IFN-I, pro-inflammatory cytokines, and cytotoxic T cell markers in tumor tissues from different treatment groups. FIG. 5F and FIG. 5G show ELISA measurement of intratumor cytokine levels in mice undergoing different treatments. Data are represented as mean±S.D. except tumor volume; *, P <0.05, **, P <0.01 (see also FIG. 11A-FIG. 11F);

FIG. 6A, FIG. 6B, FIG. 6C, FIG. 6D, FIG. 6E, and FIG. 6F show DCs primed with PSM/HER2 elicited CD8 T cell-mediated anti-tumor immunity. FIG. 6A is a Western blot analysis on MHCII protein levels in TUBO tumor tissues from post-treatment BALB/c mice. Tumor tissues were harvested 10 days after treatment. FIG. 6B shows immunohistochemical staining of MHCII in TUBO tumor tissues from post-treatment BALB/c mice; scale bar=100 μm. FIG. 6C shows immunofluorescence staining of CD11c and CD34 (vascular marker) in TUBO tumor tissues from post-treatment BALB/c mice; scale bar=50 μm. FIG. 6D shows flow cytometry analysis of CD11c- and MHCII-double-positive cells isolated from post-treatment TUBO tumor tissues. Percentage of CD11c- and MHCII-double-positive cells in each sample was labeled in red. FIG. 6E illustrates enhanced HER2-specific CD8 T cells in post-treatment TUBO tumors. In the left panel, flow cytometry analysis on CD8 and p66-pentamer positive tumor-infiltrating lymphocytes in post-treatment tumor tissues is shown. In the right panel, quantification of the percentage of p66-pentamer-positive CD8 T-cells in tumor-infiltrating lymphocytes is presented. FIG. 6F illustrates the effects of immune cell depletion on the inhibition of TUBO tumor growth by DC+PSM/p66 treatment. BALB/c mice inoculated with TUBO tumor (n=8/group) were treated with a control isotype antibody, anti-CD4 antibody, anti-CD8 antibody, or clodrosome to deplete specific cell types. Mice were then treated with DC+PSM/p66, and tumor growth was monitored in the next 4 weeks. Tumor volume is presented as mean±SEM; data are represented as mean±S.D., except tumor volume; *, P <0.05, **, P <0.01;

FIG. 7A, FIG. 7B, FIG. 7C, FIG. 7D, and FIG. 7E demonstrate that PSM potentiated antigen presentation in DCs. FIG. 7A is a cartoon illustrating the components in a DC vaccine. The antigen is packaged into liposomes and loaded into PSM particles that are then internalized by the DCs. FIG. 7B shows IL-2 production by B3Z cells after co-incubation with DCs pre-treated with different concentrations of soluble OVA or PSM/OVA. DCs were incubated with OVA or PSM/OVA for three hrs, washed extensively (without fixation), and then co-incubated with B3Z cells for 18 hrs. IL-2 levels in the cell culture medium were measured by ELISA. Data are presented as mean±S.D. FIG. 7C shows IL-2 production by B3Z cells after co-incubation with DCs pre-treated with soluble OVA, liposomal OVA, or OVA loaded in different size PSM particles. DCs were pre-treated with 100 μg/mL OVA, liposomal OVA, or PSM/OVA for three hrs, followed by fixation and extensive wash, and then co-incubated with B3Z cells. IL-2 levels in culture medium were measured with ELISA. Data are presented as mean±S.D. FIG. 7D shows intercellular transfer of PSM/antigen. DC2.4 cells were incubated with FITC-OVA—loaded PSM for 1 hr. Unbound particles were washed away, and DCs internalized with fluorescent PSM/FITC-OVA were added to a pre-seeded DC2.4 cell culture. Particle movement was tracked under a fluorescent microscope by video recording every 8 min. Snapshots demonstrating particles transferred from one cell to a neighboring cell are shown. FIG. 7E shows the in vivo release of PSM/antigen from injected DCs. BMDCs were incubated with PSM/FITC-OVA for 1 hr. After extensive washing to get rid of free particles, DCs were labeled with CellTracker™ red dye, and intravenously injected into mice. Animal tissues were harvested 24 hrs' post-injection, and frozen sections were processed and examined by fluorescence microscopy. Some green fluorescent PSM/FITC-OVA particles had separated from the red DCs in this lung tissue section; scale bar=20 μm;

FIG. 8A, FIG. 8B, and FIG. 8C show the cross-presentation of PSM/antigen in DCs. FIG. 8A illustrates IL-2 production by B3Z cells after co-incubation with WT or TAP^{-/-} DCs pretreated with OVA, PSM/OVA or peptide I (OVA 257-264, 100 ng/mL). **, P <0.01. FIG. 8B shows IL-2 production by DOBW cells after co-incubation with WT or TAP^{-/-} DCs pretreated with OVA, PSM/OVA or peptide II (OVA 323-339, 100 ng/mL). Data are presented as mean±S.D. FIG. 8C is a schematic illustration showing PSM/antigen internalization, antigen processing, and presentation from DCs to CD8 T cells;

FIG. 9A and FIG. 9B show PSM did not induce pro-inflammatory response in DCs. FIG. 9A is a quantification of IL-1β release from BMDCs 4 hrs after co-incubation with PSMs or alum. BMDCs were primed with 50 ng/mL LPS overnight prior to co-incubation with particles. Data are presented as mean±S.D.; *, P <0.05. FIG. 9B illustrates the quantitation of mRNA levels of pro-inflammatory cytokines in BMDCs 3 hrs after incubation with the indicated agents. Data are presented as mean±S.D.;

FIG. 10A, FIG. 10B, FIG. 10C, FIG. 10D, FIG. 10E, and FIG. 10F show PSM induced activation of IFN-I signaling in DCs. FIG. 10A shows quantification of TNF-α and IL-6 production by BMDCs from WT, Tlr4^{-/-l , Tlr}9^{-/-} and Tlr3^{-/-} mice 18 hrs after treatment with LPS (TLR4 ligand, 200 ng/mL), CpG (TLR9 ligand, 1 μg/mL), or poly I:C (TLR3 ligand, 20 μg/mL). Data are presented as mean±S.D.; **, P <0.01. FIG. 10B shows PSM-induced Ifn-α4 and Ifn-β gene expression in BMDCs was not affected by TLR deficiency. BMDCs isolated from wild type or knockout mice were co-incubated with PSM, and cells were harvested for IFN-I gene expression analysis. FIG. 10C, FIG. 10D, and FIG. 10E show PSM-induced Ifn-α4 and Ifn-β gene expression in BMDCs pre-treated with chemical inhibitors. Cyto-D (cytoschalatin D) is a phagocytosis inhibitor; LY-294002 is a PI3K inhibitor; and BX-795 is an inhibitor for IKKi/TBK1. FIG. 10F shows Western blot analysis on activation status of innate immune signaling pathways in DCs after treatment with medium only, antigen (Ag), PSM, or P SM/antigen;

FIG. 11A, FIG. 11B, FIG. 11C, FIG. 11D, FIG. 11E, and FIG. 11F show PSM/antigen-primed DC vaccine stimulated intratumor immunity and inhibited tumor growth. FIG. 11A is a Kaplan-Meier plot of survival of TUBO tumor-bearing mice after treatment with the indicated agents (n=8/group). This study corresponds to results in FIG. 5A. FIG. 11B shows TUBO tumor growth in mice (n=8/group) treated with PBS, DC only, or DC+PSM/TRP2. BALB/c mice were inoculated with TUBO cells in the mammary gland fat pad (0.5×10⁶ cells per mouse). Four days later, the mice were treated i.v. with PBS, DCs only, or DCs primed with PSM/TRP-2. Tumor sizes were recorded in the next 4 weeks. Tumor volume is presented as mean±SEM. FIG. 11C illustrates the percentage of intratumoral, antigen-specific CD8 T-cells in different treatment groups. BALB/c mice bearing TUBO tumors were treated with the indicated agents. Tumors were harvested 10 days after treatment, and processed for single cells. They were then stained with HER2 p66 pentamer and anti-CD8 antibody, and analyzed by flow cytometry. Percentage of p66-specific CD8 T cells in total intratumor CD8 T cells is shown in red. FIG. 11D shows the percentage of tumor-free mice in the post-treatment animals (n=5/group); this study corresponds to results in FIG. 5B. FIG. 11E is a quantitative RT-PCR analysis of Foxp3 mRNA levels in tumor tissues isolated 10 days after treatment with the indicated agents. Data are presented as mean±S.D.; *, P <0.05. FIG. 11F is an ELISA of IL-4 levels in supernatants of primary tumor cell cultures. Cells were established from tumors in different groups 10 days after treatment. Data are presented as mean±S.D.; n.s.=not statistically significant;

FIG. 12A and FIG. 12B are scanning electron microscopy (SEM) images of porous silicon microparticles (PSM). PSM are biocompatible, non-toxic, “platelet-like” microparticles that are engineered with average diameters of between about 1.0 μm and about 3.5 μm, and average thicknesses of between about 0.2 μm and about 0.7 μm. PSM can be surface-modified with 3-aminopropyl-triethoxysilane (APTES) to further enhance their stability. Unmodified PSM dissolve in water within a few hours, while APTES-conjugated PSM remain intact for up to 3 weeks;

FIG. 13 shows effective antigen presentation and activation of antigen-specific T cells facilitated by PSM. T cell response to antigen cross-presentation of PSM mixed with liposomal ovalbumin (PSM mixed with lipo-OVA) or PSM loaded with liposomal OVA (PSM loaded with lipo-OVA) were assessed in DCs using an in vitro antigen presentation assay. DCs were pulsed with agents, and then co-incubated with OVA-specific B3Z CDS T cells. IL-2 secretion by the ovalbumin-specific, CDS T cells were measured with a murine IL-2-specific ELISA kit. The result indicates that simple mixing of PSM with liposomal OVA is as effective as PSM loaded with OVA in stimulating IL-2 secretion;

FIG. 14A, FIG. 14B, FIG. 14C, and FIG. 14D show relative expression of PSM+antigen compared to PSM alone, antigen alone, or medium control in a variety of antigens;

FIG. 15A, FIG. 15B, FIG. 15C, FIG. 15D, FIG. 15E, FIG. 15F, FIG. 15G, and FIG. 1511 show relative expression of PSM alone compared to medium control in a variety of antigens;

FIG. 16 shows the change in tumor volume (mm³) over time for DC+PSM/p66, compared to DC+666, PSM+p66, or PSM alone or medium control;

FIG. 17 shows PSM potentiates adjuvant activity of the Toll-like receptor 9 agonist CpG and the STING ligand cGAMP. PSM synergizes with CpG and cGAMP on dendritic cell activation. Bone marrow-derived dendritic cells were incubated with medium only, free ovalbumin antigen (OVA), free OVA with the Toll-like receptor 9 agonist CpG and STING ligand cGAMP (CpG+cGAMP+OVA), reagents packaged in DOPC liposomes (Lipo/OVA, Lipo/CpG+OVA, Lipo/cGAMP+OVA, LIPO/CpG+cGAMP+OVA), or reagents packaged in DOPC liposomes and mixed with PSM (PSM/OVA, PSM/CpG+OVA, PSM/cGAMP+OVA, PSM/CpG+cGAMP+OVA). The dendritic cells were then incubated with OVA-specific CD8+ T cells, and IL-2 secretion from the stimulated T cells was measured. The ovalbumin class I antigen peptide (OT-I) served as the positive control in the study. A significantly increased level of IL-2 expression was detected in the PSM/CpG+cGAMP+OVA treatment group over the Lipo/CpG+cGAMP+OVA treatment group;

FIG. 18A and FIG. 18B show PSM enhances antibody production. Wild-type mice were treated by intraperitoneal injection with phosphate buffer saline control (PBS), PSM mixed with 1) liposomal OVA (PSM/OVA), 2) liposomal CpG and OVA (PSM+CpG+OVA), 3) liposomal cGAMP and OVA (PSM+cGAMP+OVA), 4) liposomal CpG and cGAMP and OVA (PSM/CpG+cGAMP+OVA), or aluminum oxide microparticles mixed with OVA (Alum+OVA). Plasma OVA-specific antibody levels were measured 1, 2, and 3 weeks later. Data from week 3 samples are shown for IgG2a (FIG. 18A) and IgG2b (FIG. 18B) titers. The trends were the same for samples from both week 1 and week 2. Alum serves as the positive control in the study, as it is commonly used as an adjuvant for antibody-based vaccines. The results indicated that PSM synergizes with CpG and cGAMP on antibody production;

FIG. 19A, FIG. 19B, FIG. 19C, FIG. 19D, and FIG. 19E show PSM synergizes with CpG and cGAMP on memory T cell production. BALB/c mice bearing HER2-positive TUBO tumors treated with dendritic cells pulsed with: PBS control (FIG. 19A); PSM mixed with liposomal p66 antigen (PSM/p66) (FIG. 19B); PSM mixed with liposomal CpG+p66 (PSM/CpG+p66) (FIG. 19C); PSM mixed with cGAMP+p66 (PSM/cGAMP+p66) (FIG. 19D); or PSM mixed with CpG+cGAMP+p66 (PSM/CpG+cGAMP+p66) (FIG. 19E). Mice were euthanized 6 weeks after vaccination, and single cells were isolated from the tumor tissues and analyzed for CD44A positive memory T cells. The result shows dramatically increased memory T cell level in mice treated with PSM/CpG+cGAMP+p66 over the other groups;

FIG. 20 shows the stimulation of antibody production against the West Nile Virus protein in wild-type mice treated with PSM/(CpG+cGAMP+WNV); and

FIG. 21 shows nanovaccine treatment inhibits HER2 positive breast cancer. BALB/c mice were inoculated with HER2-expressing TUBO tumors. Mice were inoculated with indicated vaccines once when tumor size reached 100 mm³ size and another time 7 days later.

### BRIEF DESCRIPTION OF THE SEQUENCES:

SEQ ID NO:1 is an exemplary peptide for use in accordance with one aspect of the present disclosure.

SEQ ID NO:2 is an exemplary peptide for use in accordance with one aspect of the present disclosure.

### DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

Cancer immunotherapy has gained a high level of attention recently, thanks in part to recent approval by the United States Food and Drug Administration (FDA) of a dendritic cell-based therapy for metastatic prostate cancer (sipuleucel-T; PROVENGE®, Dendreon Corp., Seattle, Wash., USA) and clinical checkpoint blockade antibody-based therapies for late-stage cancer treatment [e.g., anti-CTLA4 (ipilimumab, YERVOY® and nivolumab, OPDIVO®, Bristol-Myers Squibb, Princeton, N.J., USA) and anti-PD1 (MK-3475, pembrolizumab, KEYTRUDA®, Merck Oncology, Whitehouse Junction, N.J., USA) (see e.g., Hodi et al., 2010; Postow et al., 2015; Sharma et al., 2011). Despite these advances, complete response to cancer immunotherapy remains sparse in clinic due to multiple factors such as inefficient vaccine delivery, defective antigen cross presentation in tumor tissues, infiltration of suppressive immune cells including regulatory T cells (Tregs) and myeloid-derived suppressor cells (MDSCs), and immunosuppressive cytokine milieu (Dougan and Dranoff, 2009). Approaches to reverse the immunosuppressive tumor microenvironment are anticipated to have a significant impact on cancer immunotherapy (Gajewski et al., 2013b).

Innate immunity is a major component of tumor immunity, and proper activation of innate immune cells by recognizing tumor antigens and danger signals from tumor cells ensures efficient adaptive immunity against cancer (Dougan and Dranoff, 2009). Thus, factors bridging innate immunity and adaptive immunity can be targeted for cancer immunotherapy. Dendritic cells (DCs) are the professional antigen presenting cells by surveying and processing antigen to T cells, and the antigen presentation process often requires subcellular antigen delivery and innate immune signaling. It has been previously reported that class I antigen is processed in early endosome and the Toll-like receptor 4 (TLR4)-MyD88 activity is required for proper relocation of the transporter associated with antigen processing (Burgdorf et al., 2008). However, that study was performed on soluble antigen cross presentation; whether the same mechanism can be applied to other forms of antigens (such as particulate antigens) remains unknown.

Innate immune stimuli such as TLR ligands often serve as immune adjuvants to enhance DC-based immune responses (Coffman et al., 2010). TLR activation stimulates downstream pathways such as NF-κB signaling, and MAPK signaling for pro-inflammatory cytokine induction (Kawai and Akira, 2011). These cytokines will further induce expression and translocation of antigen presenting molecules and promote antigen processing. Ironically, too strong TLR stimulation may induce detrimental inflammatory responses (Spaner et al., 2008), a fact which prevents their use in clinic. Besides inflammatory cytokines, type I interferons (IFN-I) also promote DC maturation, antigen cross-presentation, and CD8 T-cell clonal expansion (Coffman et al., 2010; Le Bon and Tough, 2008). Furthermore, a recent study reported a pivotal role of IFN-I in anti-tumor immunity by reactivating cross presentation function in intratumor DCs (Yang et al., 2014).

Physical properties of antigens and adjuvants may contribute to their immune-stimulating functions. The size, shape, and surface characteristics of an antigen or adjuvant have a significant impact on its immunogenicity (Bachmann and Jennings, 2010). Particulate antigen vaccine might provide advantage over the soluble antigen vaccine by serving as antigen depot and protecting the antigen from enzyme degradation, enabling targeted delivery to specific immune organs and cell types, and stimulating antigen presentation via the desired pathways at controlled release rate (Paulis et al., 2013). For example, alum adjuvant and many nano-size crystal structures can activate inflammasome and promote IL-1β release in DCs, which may facilitate the antigen presentation function of DCs and boost immune responses (Sharp et al., 2009). Nevertheless, the mechanism of action of these particles is still not well understood.

Micrometer—and nanometer-sized particles have become popular candidates for cancer vaccine adjuvants in recent years. However, the mechanism by which such particles enhance immune responses remains unclear.

Porous silicon microparticles (PSMs) are micrometer-sized, discoid-shaped silicon particles containing nanopores that can carry nano-sized drugs, and have been used for delivery of small molecule drugs and other cancer therapeutics (Chen et al., 2014; Dave et al., 2014; Shen et al., 2013a; Xu et al., 2013). This drug carrier is degradable and biocompatible, and the rate of release of the cargo can be tailored by surface chemical modification (Shen et al., 2013b; Tanaka et al., 2010; Xu et al., 2013). Here, the potential of PSM as an adjuvant for cancer vaccine was examined. PSMs loaded with liposomal antigen inside the nanopores were efficiently internalized by DCs and trafficked to early endosomes for efficient cross presentation. Notably, phagocytosis of PSM induced type I interferon responses in DCs. In addition, DCs primed with PSM-loaded HER2 antigen peptide inhibited growth of HER2-overexpressing mammary gland tumors in mice by stimulating stroma cell MHCII expression, Th1 cytokine production, and intratumor CD8 T cell cytotoxicity. Thus, PSM represents a new adjuvant to DC-based immunotherapy by improving cross presentation and modulating local immune microenvironment.

Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. The following references provide one of skill with a general definition of many of the terms used in this invention: Dictionary of Biochemistry and Molecular Biology, (2^{nd} Ed.) J. Stenesh (Ed.), Wiley-Interscience (1989); Dictionary of Microbiology and Molecular Biology (3^{rd} Ed.), P. Singleton and D. Sainsbury (Eds.), Wiley-Interscience (2007); Chambers Dictionary of Science and Technology (2^{nd} Ed.), P. Walker (Ed.), Chambers (2007); Glossary of Genetics (5^{th} Ed.), R. Rieger et al. (Eds.), Springer-Verlag (1991); and The HarperCollins Dictionary of Biology, W. G. Hale and J. P. Margham, (Eds.), HarperCollins (1991).

Although any methods and compositions similar or equivalent to those described herein can be used in the practice or testing of the present invention, the preferred methods, and compositions are described herein. For purposes of the present invention, the following terms are defined below for sake of clarity and ease of reference:

In accordance with long standing patent law convention, the words “a” and “an,” when used in this application, including the claims, denote “one or more.”

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic.

“Biocompatible” refers to a material that, when exposed to living cells, will support an appropriate cellular activity of the cells without causing an undesirable effect in the cells, such as a change in a living cycle of the cells, a change in a proliferation rate of the cells, or a cytotoxic effect.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, the term “DNA segment” refers to a DNA molecule that has been isolated free of total genomic DNA of a particular species. Therefore, a DNA segment obtained from a biological sample using one of the compositions disclosed herein refers to one or more DNA segments that have been isolated away from, or purified free from, total genomic DNA of the particular species from which they are obtained. Included within the term “DNA segment,” are DNA segments and smaller fragments of such segments, as well as recombinant vectors, including, for example, plasmids, cosmids, phage, viruses, and the like.

The term “effective amount,” as used herein, refers to an amount that is capable of treating or ameliorating a disease or condition or otherwise capable of producing an intended therapeutic effect.

As used herein, the term “epitope” refers to that portion of a given immunogenic substance that is the target of, i.e., is bound by, an antibody or cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art (see, for example, Geysen et al., 1984). An epitope can be a portion of any immunogenic substance, such as a protein, polynucleotide, polysaccharide, an organic or inorganic chemical, or any combination thereof. The term “epitope” may also be used interchangeably with “antigenic determinant” or “antigenic determinant site.”

The term “for example” or “e.g.,” as used herein, is used merely by way of example, without limitation intended, and should not be construed as referring only those items explicitly enumerated in the specification.

As used herein, the term “homology” refers to a degree of complementarity between two polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

As used herein, “homologous” means, when referring to polypeptides or polynucleotides, sequences that have the same essential structure, despite arising from different origins. Typically, homologous proteins are derived from closely related genetic sequences, or genes. By contrast, an “analogous” polypeptide is one that shares the same function with a polypeptide from a different species or organism, but has a significantly different form to accomplish that function. Analogous proteins typically derive from genes that are not closely related.

As used herein, the phrase “in need of treatment” refers to a judgment made by a caregiver such as a physician or veterinarian that a patient requires (or will benefit in one or more ways) from treatment. Such judgment may made based on a variety of factors that are in the realm of a caregiver's expertise, and may include the knowledge that the patient is ill as the result of a disease state that is treatable by one or more compound or pharmaceutical compositions such as those set forth herein.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

The phrases “isolated” or “biologically pure” refer to material that is substantially, or essentially, free from components that normally accompany the material as it is found in its native state.

As used herein, the term “kit” may be used to describe variations of the portable, self-contained enclosure that includes at least one set of reagents, components, or pharmaceutically-formulated compositions to conduct one or more of the assay methods of the present invention. Optionally, such kit may include one or more sets of instructions for use of the enclosed compositions, such as, for example, in a laboratory or clinical application.

“Link” or “join” refers to any method known in the art for functionally connecting peptides, including, without limitation, recombinant fusion, covalent bonding, disulfide bonding, ionic bonding, hydrogen bonding, electrostatic bonding, and such like.

“Microparticle” means a particle having a maximum characteristic size from 1 micron to 1000 microns or from 1 micron to 100 microns. Preferably, the porous particle of this disclosure should have a relatively high porosity to enable loading of the polymeric-active agent conjugate in the pores of the porous particles. Optionally, the porous particles of the present disclosure may be coated with a targeting moiety. Such embodiments may be useful for targeted delivery of the active compound to the desired disease site.

“Nanoparticle” means a particle having a maximum characteristic size of less than 1 micron. Preferably, the polymeric-active agent conjugate of this disclosure forms nanoparticles upon release from the porous silicon particle upon physiological degradation of the porous particle, and upon coming in contact with an aqueous environment.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases, and intronic sequences may be of variable lengths; some polynucleotide elements may be operably linked, but not contiguous.

As used herein, the term “patient” (also interchangeably referred to as “host” or “subject”), refers to any host that can serve as a recipient of one or more of the therapeutic or diagnostic formulations as discussed herein. In certain aspects, the patient is a vertebrate animal, which is intended to denote any animal species (and preferably, a mammalian species such as a human being). In certain embodiments, a patient may be any animal host, including but not limited to, human and non-human primates, avians, reptiles, amphibians, bovines, canines, caprines, cavines, corvines, epines, equines, felines, hircines, lapines, leporines, lupines, murines, ovines, porcines, racines, vulpines, and the like, including, without limitation, domesticated livestock, herding or migratory animals or birds, exotics or zoological specimens, as well as companion animals, pets, or any animal under the care of a veterinary or animal medical care practitioner.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′ dibenzylethylenediamine or ethylenediamine; and combinations thereof.

The term “pharmaceutically acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia.

As used herein, the term “plasmid” or “vector” refers to a genetic construct that is composed of genetic material (i.e., nucleic acids). Typically, a plasmid or a vector contains an origin of replication that is functional in bacterial host cells, e.g., Escherichia coli, and selectable markers for detecting bacterial host cells including the plasmid. Plasmids and vectors of the present invention may include one or more genetic elements as described herein arranged such that an inserted coding sequence can be transcribed and translated in a suitable expression cells. In addition, the plasmid or vector may include one or more nucleic acid segments, genes, promoters, enhancers, activators, multiple cloning regions, or any combination thereof, including segments that are obtained from or derived from one or more natural and/or artificial sources.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “L” isomeric form. However, residues in the “D” isomeric form may be substituted for any L-amino acid residue provided the desired properties of the polypeptide are retained.

As used herein, the terms “prevent,” “preventing,” “prevention,” “suppress,” “suppressing,” and “suppression” as used herein refer to administering a compound either alone or as contained in a pharmaceutical composition prior to the onset of clinical symptoms of a disease state so as to prevent any symptom, aspect or characteristic of the disease state. Such preventing and suppressing need not be absolute to be deemed medically useful.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about two to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. Polypeptides and proteins of the present invention also include polypeptides and proteins that are or have been post-translationally modified, and include any sugar or other derivative(s) or conjugate(s) added to the backbone amino acid chain.

“Purified,” as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “RNA segment,” as used herein, refers to an RNA molecule that has been isolated free of total cellular RNA of a particular species. Therefore, RNA segments can refer to one or more RNA segments (either of native or synthetic origin) that have been isolated away from, or purified free from, other RNAs. Included within the term “RNA segment,” are RNA segments and smaller fragments of such segments.

The term “sequence,” when referring to amino acids, relates to all or a portion of the linear N-terminal to C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide chains, “sequence” and “subsequence” have similar meanings relating to the 5′ to 3′ order of nucleotides.

As used herein, the term “structural gene” is intended to generally describe a polynucleotide, such as a gene, that is expressed to produce an encoded peptide, polypeptide, protein, ribozyme, catalytic RNA molecule, or antisense molecule.

The term “a sequence essentially as set forth in SEQ ID NO:X” means that the sequence substantially corresponds to a portion of SEQ ID NO:X and has relatively few nucleotides (or amino acids in the case of polypeptide sequences) that are not identical to, or a biologically functional equivalent of, the nucleotides (or amino acids) of SEQ ID NO:X. The term “biologically functional equivalent” is well understood in the art, and is further defined in detail herein. Accordingly, sequences that have about 85% to about 90%; or more preferably, about 91% to about 95%; or even more preferably, about 96% to about 99%; of nucleotides that are identical or functionally equivalent to one or more of the nucleotide sequences provided herein are particularly contemplated to be useful in the practice of the invention.

Suitable standard hybridization conditions for the present invention include, for example, hybridization in 50% formamide, 5× Denhardt's solution, 5× SSC, 25 mM sodium phosphate, 0.1% SDS and 100 μg/mL of denatured salmon sperm DNA at 42° C. for 16 hr followed by 1 hr sequential washes with 0.1× SSC, 0.1% SDS solution at 60° C. to remove the desired amount of background signal. Lower stringency hybridization conditions for the present invention include, for example, hybridization in 35% formamide, 5× Denhardt's solution, 5× SSC, 25 mM sodium phosphate, 0.1% SDS and 100 μg/mL denatured salmon sperm DNA or E. coli DNA at 42° C. for 16 hr followed by sequential washes with 0.8× SSC, 0.1% SDS at 55° C. Those of skill in the art will recognize that conditions can be readily adjusted to obtain the desired level of stringency.

Naturally, the present invention also encompasses nucleic acid segments that are complementary, essentially complementary, and/or substantially complementary to at least one or more of the specific nucleotide sequences specifically set forth herein. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to one or more of the specific nucleic acid segments disclosed herein under relatively stringent conditions such as those described immediately above.

As described above, the probes and primers of the present invention may be of any length. By assigning numeric values to a sequence, for example, the first residue is 1, the second residue is 2, etc., an algorithm defining all probes or primers contained within a given sequence can be proposed:
n to n+y,

where n is an integer from 1 to the last number of the sequence and y is the length of the probe or primer minus one, where n+y does not exceed the last number of the sequence. Thus, for a 25-basepair probe or primer (i.e., a “25 mer”), the collection of probes or primers correspond to bases 1 to 25, bases 2 to 26, bases 3 to 27, bases 4 to 28, and so on over the entire length of the sequence. Similarly, for a 35-basepair probe or primer (i.e., a “35-mer), exemplary primer or probe sequence include, without limitation, sequences corresponding to bases 1 to 35, bases 2 to 36, bases 3 to 37, bases 4 to 38, and so on over the entire length of the sequence. Likewise, for 40-mers, such probes or primers may correspond to the nucleotides from the first basepair to bp 40, from the second bp of the sequence to bp 41, from the third bp to bp 42, and so forth, while for 50-mers, such probes or primers may correspond to a nucleotide sequence extending from bp 1 to bp 50, from bp 2 to bp 51, from bp 3 to bp 52, from bp 4 to bp 53, and so forth.

The term “subject,” as used herein, describes an organism, including mammals such as primates, to which treatment with the compositions according to the present invention can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, about 2, about 3, about 4, about 5, about 6, about 7, about 8, about 9, or about 10 or so base mismatches throughout the complementary portion of the sequence. In many instances, it may be desirable for the sequences to be exact matches, i.e., be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary nucleic acid sequences will be greater than about 80 percent complementary (or “% exact-match”) to a corresponding nucleic acid target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including about 96%, about 97%, about 98%, about 99%, and even about 100% exact match complementary to all or a portion of the target sequence to which the designed nucleic acid specifically binds.

Percent similarity or percent complementary of any of the disclosed nucleic acid sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

As used herein, “synthetic” shall mean that the material is not of a human or animal origin.

The term “therapeutically practical time period” means a period of time that is necessary for an active agent to be therapeutically-effective. The term “therapeutically-effective” refers to reduction in severity and/or frequency of symptoms, elimination of symptoms and/or underlying cause, prevention of the occurrence of symptoms and/or their underlying cause, and improvement or remediation of damage.

A “therapeutic agent” may be any physiologically or pharmacologically active substance that may produce a desired biological effect in a targeted site in a subject. The therapeutic agent may be a chemotherapeutic agent, an immunosuppressive agent, a cytokine, a cytotoxic agent, a nucleolytic compound, a radioactive isotope, a receptor, and a pro-drug activating enzyme, which may be naturally-occurring, or produced by synthetic or recombinant methods, or any combination thereof. Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) may have particular utility as the therapeutic agent. Cytokines may be also used as the therapeutic agent. Examples of such cytokines are lymphokines, monokines, and traditional polypeptide hormones. A cancer chemotherapy agent may be a preferred therapeutic agent. For a more detailed description of anticancer agents and other therapeutic agents, those skilled in the art are referred to any number of instructive manuals including, but not limited to, the Physician's Desk Reference and to Goodman and Gilman's “Pharmacological Basis of Therapeutics” tenth edition, Hardman et al. (Eds.) (2001).

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s), which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted based on known consensus sequence motifs, or by other methods known to those of skill in the art.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

As used herein, the term “transformed cell” is intended to mean a host cell whose nucleic acid complement has been altered by the introduction of one or more exogenous polynucleotides into that cell.

As used herein, the term “transformation” is intended to generally describe a process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

“Treating” or “treatment of” as used herein, refers to providing any type of medical or surgical management to a subject. Treating can include, but is not limited to, administering a composition comprising a therapeutic agent to a subject. “Treating” includes any administration or application of a compound or composition of the invention to a subject for purposes such as curing, reversing, alleviating, reducing the severity of, inhibiting the progression of, or reducing the likelihood of a disease, disorder, or condition or one or more symptoms or manifestations of a disease, disorder, or condition. In certain aspects, the compositions of the present invention may also be administered prophylactically, i.e., before development of any symptom or manifestation of the condition, where such prophylaxis is warranted. Typically, in such cases, the subject will be one that has been diagnosed for being “at risk” of developing such a disease or disorder, either as a result of familial history, medical record, or the completion of one or more diagnostic or prognostic tests indicative of a propensity for subsequently developing such a disease or disorder.

The expression “zero-order or near-zero-order” as applied to the release kinetics of the active agent delivery composition disclosed herein is intended to include a rate of release of the active agent in a controlled manner over a therapeutically practical time period following administration of the composition, such that a therapeutically effective plasma concentration of the active agent is achieved.

In certain embodiments, it will be advantageous to employ one or more nucleic acid segments of the present invention in combination with an appropriate detectable marker (i.e., a “label,”), such as in the case of employing labeled polynucleotide probes in determining the presence of a given target sequence in a hybridization assay. A wide variety of appropriate indicator compounds and compositions are known in the art for labeling oligonucleotide probes, including, without limitation, fluorescent, radioactive, enzymatic or other ligands, such as avidin/biotin, etc., which are capable of being detected in a suitable assay. In particular embodiments, one may also employ one or more fluorescent labels or an enzyme tag such as urease, alkaline phosphatase or peroxidase, instead of radioactive or other environmentally less-desirable reagents. In the case of enzyme tags, colorimetric, chromogenic, or fluorogenic indicator substrates are known that can be employed to provide a method for detecting the sample that is visible to the human eye, or by analytical methods such as scintigraphy, fluorimetry, spectrophotometry, and the like, to identify specific hybridization with samples containing one or more complementary or substantially complementary nucleic acid sequences. In the case of so-called “multiplexing” assays, where two or more labeled probes are detected either simultaneously or sequentially, it may be desirable to label a first oligonucleotide probe with a first label having a first detection property or parameter (for example, an emission and/or excitation spectral maximum), which also labeled a second oligonucleotide probe with a second label having a second detection property or parameter that is different (i.e., discreet or discernible from the first label. The use of multiplexing assays, particularly in the context of genetic amplification/detection protocols are well-known to those of ordinary skill in the molecular genetic arts.

The section headings used throughout are for organizational purposes only and are not to be construed as limiting the subject matter described. All documents, or portions of documents, cited in this application, including, but not limited to, patents, patent applications, articles, books, and treatises, are incorporated herein in their entirety by express reference thereto. In the event that one or more of the incorporated literature and similar materials defines a term in a manner that contradicts the definition of that term in this application, this application controls.

### EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure, appreciate that many changes can be made in the specific embodiments that are disclosed, and still obtain a like or similar result without departing from the spirit and scope of the invention.

### Example 1

### PSM Potentiate Antitumoral Immunity by Enhancing Cross-Presentation and Inducing Type I Interferon

In this Example, a PSM-based cancer vaccine is described that greatly enhanced cross-presentation and activated type I interferon response in dendritic cells. PSM-loaded antigen exhibited prolonged early endosome localization and enhanced cross-presentation through both proteasome- and lysosome-dependent pathways. Phagocytosis of PSM by dendritic cells induced type I interferon responses through TRIF- and MAVS-dependent pathways. Dendritic cells primed with PSM-loaded HER2 antigen produced robust CD8 T cell-dependent anti-tumor immunity in mice bearing HER2-positive mammary gland tumors. Importantly, this vaccination activated tumor immune microenvironment with elevated levels of intratumor type I interferon and MHC-II expression, abundant CD11c+ dendritic cell infiltration, and tumor-specific cytotoxic T cell responses. These findings highlight the use of PSM-based adjuvants to potentiate dendritic cell-based cancer immunotherapy.

Materials and Methods

Cells and Mice. The immature C57BL/6 DC line, DC2.4, was obtained from The University of Massachusetts Medical School (Worcester, Mass., USA). DOBW cells, a T-T hybridoma against OVA (323-339)/I-A^{d} complex, were obtained from Case Western Reserve University (Cleveland, Ohio, USA). B3Z cells, T-T hybridomas against OVA (257-264)/H-2K^{b} complex, were obtained from the University of California-Berkeley (Berkeley, Calif., USA). All DC and T-cell lines were maintained in RPMI 1640 medium containing 10% FBS, 1 mM pyruvate, 2 mM L-glutamine, 50 μM 2-mercaptoethanol, and penicillin/streptomycin (100 μg/mL each). TUBO cells and BALB-neuT transgenic mice were obtained from University of Turin (Turin, ITALY) and The Mayo Clinic (Rochester, Minn., USA). C57BL/6 and BALB/c mice were obtained from Charles River (Wilmington, Mass., USA) or The Jackson Laboratory (Bar Harbor, Me., USA). B6.129S2-Tap1^{tm1Arp}/J (Tap^{-/-}), B6N.129S1-Tlr3^{tm1flv}/J (Tlr3^{-/-}), C57BL/6J-Ticam1^{LPS 2}/J (Trit), C57BL/6J-Tmem173^{gt}/J (Sting^{Gt/Gt}) mouse lines were obtained from The Jackson Laboratory. Tlr4^{-/-}, Tlr9^{-/-}, and Myd88^{-/-} mouse lines were maintained as previously described (Duggan et al., 2011; Millien et al., 2013). All animal studies were performed using institutional-approved guidelines.

Preparation of Antigen-Encapsulated Liposomes and PSM Loading. Liposomes were made as described previously (Tanaka et al., 2010; Xu et al., 2013). Briefly, proteins or peptide antigens were dissolved in H₂O; 1,2-dioleoyl-sn-glycero-3-phosphocholine (DOPC) was prepared in tent-butanol at 20 mg/mL, and 0.1% Tween-20® was prepared in water, then mixed together into tent-butanol by vortexing for 1 min. Samples were then freeze-dried in a lyophilizer. To load the liposomes into PSM particles, 120 μL H₂O was added to liposome powder and followed by brief sonication. The liposomes in H₂O were then added to dry PSM particles, followed by brief sonication.

In Vitro Antigen Presentation Assay. The in vitro antigen presentation assay was performed as previously described (Mukai et al., 2011). Briefly, DC2.4 cells or BMDCs isolated from C57BL/6 mice or TAP^{-/-} mice were seeded in a 96-well flat-bottom culture plates at a density of 2×10⁵ cells/well, and cultured for 12 hrs at 37° C. Each well was washed once with PBS, and then the cells were pulsed with soluble OVA, PSM/OVA, OVA-derived MHC class I epitope peptide [OVA257-264; SIINFEKL (SEQ ID NO:1)], or MHC class II epitope peptide [OVA323-339; ISQAVHAAHAEINEAGR (SEQ ID NO:2); Peptide 2.0] for indicated time points, and immediately fixed with 1% paraformaldehyde. The cells were then co-cultured with 2×10⁵ B3Z cells or 2×10⁵ DOBW cells for 18 hrs at 37° C. The response of stimulated B3Z or DOBW cells was assessed by determining the amount of IL-2 released into the culture medium using a murine IL-2 ELISA KIT (Ebioscience, San Diego, Calif., USA). For the inhibition assay, BMDCs were pretreated with 10 mM MG-132 (EMD Chemicals, San Diego, Calif., USA), 1 mM epoxomicin (EMD Chemicals), or 40 mM leupeptin (EMD Chemicals) for 1 hr at 37° C. before antigen pulsing. The antigen pulsing media also contained each inhibitor.

Confocal Laser Scanning Microscopy. DC2.4 cells or BMDCs were seeded on eight-well chamber slides at a density of 1×10⁵ cells/well and cultured for 24 hrs at 37° C. Each well was washed twice with PBS, and the cells were pulsed with FITC-OVA or PSM/FITC-OVA for 20 min. Cells were washed twice with PBS and incubated for an additional 30 min, 1, 3, or 6 hours in culture medium at 37° C. Cells were washed with PBS, fixed with 4% paraformaldehyde, and then stained with antibodies against specific markers of individual organelles. Antibodies used to detect the early endosome, late endosome, early and recycling endosome, and endoplasmic reticulum (ER) were rabbit anti-EE Ag 1 (EEA1) Ab (ab74906; Abcam, Cambridge, Mass., USA), rabbit anti-Rab7 Ab (ab50313; Abcam), rat anti-transferrin receptor (TfR) Ab (ab22391; Abcam), and rabbit anti-KDEL Ab (ab50601; Abcam), respectively. Cells were then washed with PBS, and incubated with Alexa 594-conjugated anti-rabbit IgG Ab (A11037; Invitrogen, Carlsbad, Calif., USA) or Alexa 594-conjugated anti-rat IgG Ab (A21209; Invitrogen) for 1 hr at room temperature. The samples were embedded with Prolong® Gold anti-fade reagent with DAPI (Invitrogen) and analyzed by confocal laser scanning microscopy (Olympus IX81).

Transmission Electron Microscopy. Transmission electron microscopy (TEM) analysis was performed by the TEM core facility at MD Anderson Cancer Center (Houston, Tex., USA). BMDCs isolated from C57BL/6 mice were seeded on 24-well plates at a density of 1×10⁶ cells and cultured for 24 hrs at 37° C. The culture dish was washed twice with PBS, and then cells were pulsed with PSM/OVA for 20 min at 37° C. Cells were washed twice with PBS and incubated for an additional two hrs in culture medium at 37° C. Cells were fixed in 4% paraformaldehyde. After fixation, samples were washed and treated with 0.1% cacodylate-buffered tannic acid, post-fixed with 1% buffered osmium tetroxide for 30 min, and stained with 1% uranyl acetate. Samples were then dehydrated in increasing concentration of ethanol and embedded in Poly/Bed® 812 resin (Ted Pella, Inc., Redding, Calif., USA). Samples were polymerized in a 60° C. oven for 2 days. Ultrathin sections were cut using an ultramicrotome (Ultracut®; Leica Biosystems, Deerfield, Ill., USA), stained with uranyl acetate and lead citrate in a EM Stainer (Leica Biosystems), and examined in a transmission electron microscope (JEM1010@; JEOL USA, Inc., Peabody, Mass., USA).

Real-Time PCR. Total RNA was isolated with Trizol reagent (Life Technologies, Inc. Grand Island, N.Y., USA) and used for cDNA synthesis with a reverse transcription kit (Roche, Nutley, N.J., USA). Real-time PCR was performed with SYBR® green select Master Mix on a StepOnePlus™ Real-Time PCR System (Life Technologies). The expression of individual genes was calculated by a ΔΔCT method, and normalized to the expression of β-actin.

Tumor Studies. TUBO cells (1×10⁶ cells/mouse) suspended in cold Matrigel™/PBS (1:1) were implanted into mammary gland fat pads of female BALB/c mice (aged 6-10 weeks). On Day 4 after tumor inoculation (tumor reached palpable size), animals were randomly divided into groups (eight mice per group) and received i.v. injection of treatments. The mice were monitored for tumor size, and survival. Tumor volume was recorded as width×length²/2. For intratumoral cytokine and protein analyses, mice were sacrificed on Day 14 after tumor inoculation and tumors were harvested for immune cell typing, RNA and protein analysis by flow cytometry, qPCR, and Western blot analysis, respectively. In a tumor prevention study, using BALB-neuT mice, groups of mice (five mice per group) were treated by i.v. injection of vaccine at ages 6 and 8 weeks, and then the tumor incidence, tumor multiplicity, and mouse survival was monitored for 30 weeks. In the depletion studies, anti-CD4 (GK1.5) and anti-CD8 (2.43) mAbs were used for in vivo depletion of T-cell subsets. Clodrosomes (Encapsula NanoSciences, LLC, Brentwood, Tenn., USA) were used to deplete macrophages. Animals were injected i.p. with 200 μg of anti-CD4 and anti-CD8 mAb or 200 μL of clodrosomes twice per week for 3 weeks, starting 1 week before inoculation of the tumor cells (0.5×10⁶ cells per mouse).

Statistical Analyses. Statistical significance was determined by Student's t-test to evaluate the P-value. The relationship between two variables was tested using regression analysis, and P <0.05 was considered significant. Survival analysis was analyzed by the Log-rank test to compare Kaplan-Meier survival curves.

BMDCs. 6-10 week old female mice were used to isolate bone marrow-derived DCs. Briefly, femur and tibia from mice hind legs were collected and bone marrow cells were flushed out with 1% FBS-containing PBS using a syringe. Cells were treated briefly with ACK lysis buffer (Lonza, Inc., Basel, SWITZERLAND) to remove red blood cells, and then resuspended into RPMI1640 medium with 10% FBS, antibiotics, 55 μM 2-mercaptoethanol. Cells were grown with supplement of recombinant murine GM-CSF and IL-4 (20 ng/mL). Cell culture medium was refreshed every other day. Non-adherent cells were harvested as immature BMDCs. For in vivo studies, BMDCs were stimulated with 100 ng/mL LPS overnight for maturation, and then primed with soluble antigen or PSM/antigen for three hrs before i.v. injection into mice.

Cytokine Measurement. Cytokine release from cell culture was measured by ELISA using Bio-Plex Pro™ Mouse Cytokine 23-plex Assay (Bio-Rad Laboratories, Inc., Hercules, Calif., USA) or individual cytokine ELISA kits from Ebioscience or PBL Assay Science (Piscataway, N.J., USA).

shRNA-Mediated Gene Knockdown. pLKO.1 lentiviral vectors encoding shRNAs were described previously (Zhang et al., 2011), or purchased from Sigma-Aldrich Chemical Co. (St. Louis, Mo., USA). To produce lentiviral particles, the lentiviral vectors were transfected into HEK293T cells along with the packaging plasmids, psPAX2 and pMD2. Dendritic cells were infected with the lentiviruses as previously described (Zhang et al., 2011), and knockdown efficiency was confirmed by real-time PCR.

Results and Discussion

Uptake of PSM-Loaded Antigens by Dendritic Cells. Particle uptake by immune cells was analyzed in mice treated with intravenous injection of PSMs, and it was shown that the particles were preferentially engulfed by CD11c+ DCs over F4/80+ macrophages in peripheral blood (see Table 1).

TABLE 1
IN VIVO UPTAKE OF PSMS BY IMMUNE
CELLS IN MOUSE PERIPHERAL BLOOD.

| Cell Type | 0 min | 10 min | 60 min |
|---|---|---|---|
| Immature monocytes | 0% | 3.21% | 7.24% |
| Mature monocytes | 0% | 1.36% | 1.81% |
| Neutrophils | 0% | 0.57% | 0.64% |
| Dendritic cells | 0% | 4.48% | 12.33% |
| Macrophages | 0% | 1.2% | 1.35% |

Percentage uptake was calculated as number of PSM positive cells in total number of the specified cell type (CD115⁺Ly6C^{high} Immature monocytes, CD115⁺Ly6C^{low} mature monocytes, Gr1⁺CD11b⁺ neutrophils, F4/80⁺CD11b⁻ macrophages, CD11c⁺CD11b⁺ dendritic cells) at different time points after intravenous administration of PSM. One of two representative studies is shown.

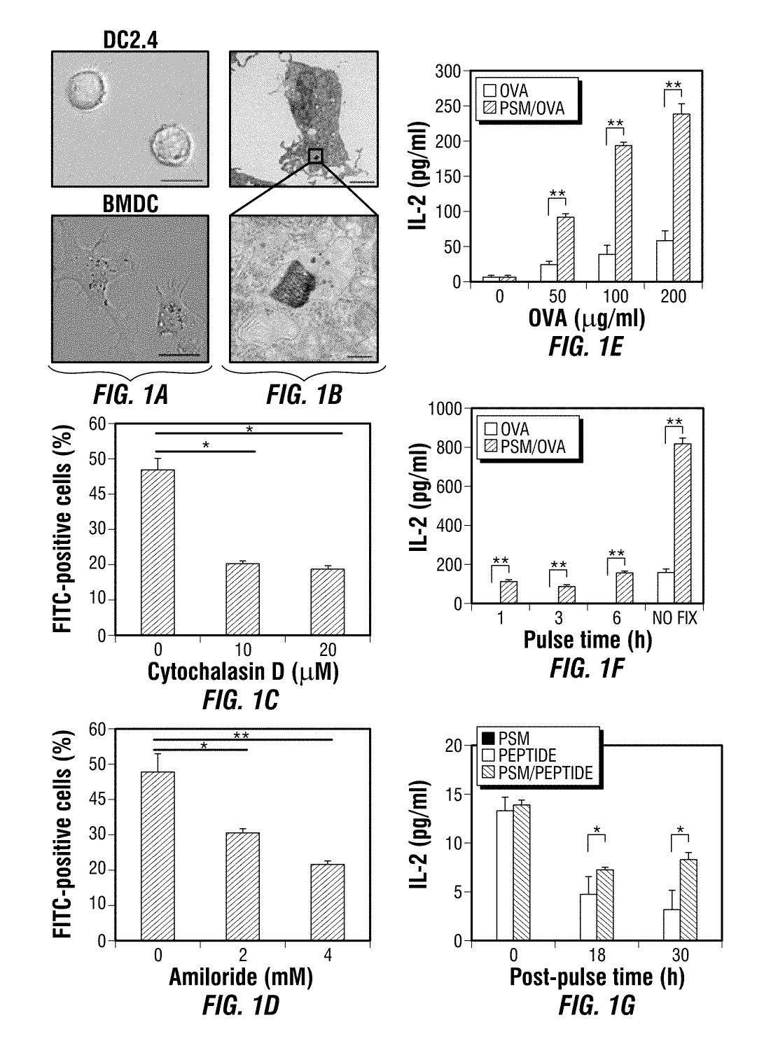

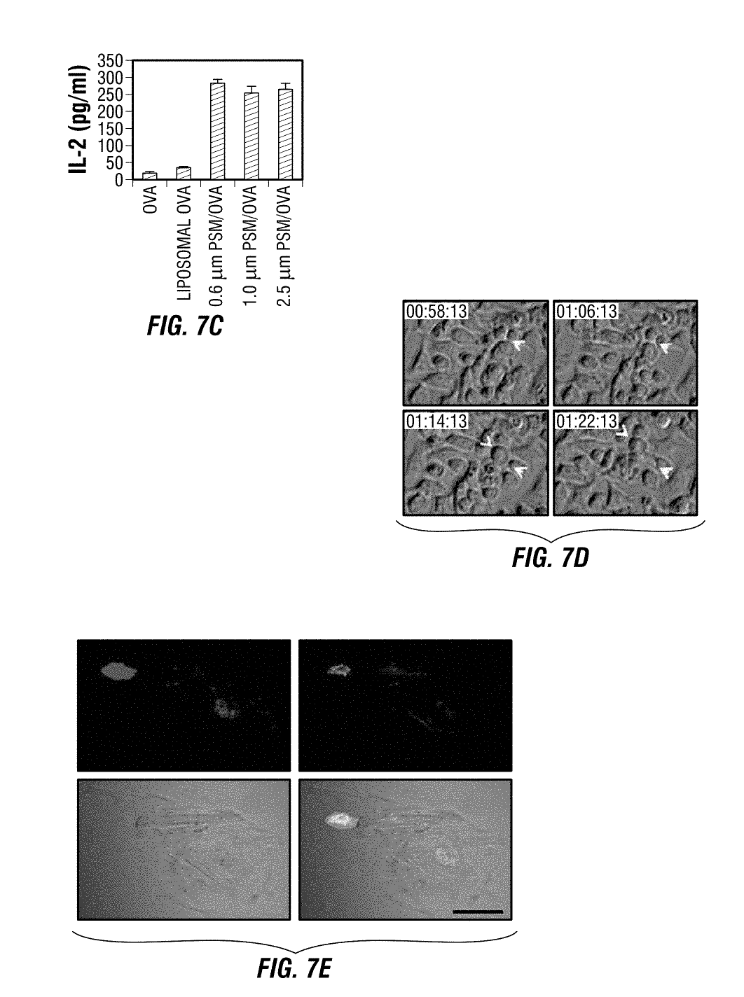

Since DCs are the most efficient antigen presenting cells, the possibility of applying PSMs to potentiate antigen presentation was explored. Ovalbumin (OVA), a well-characterized model antigen, was first applied for in vitro and in vivo immune response studies (FIG. 7A). FITC-conjugated OVA (FITC-OVA) was packaged into liposomes, and loaded into PSM following a previously described procedure (Shen et al., 2013a; Tanaka et al., 2010; Xu et al., 2013). The PSM-loaded FITC-OVA (PSM/FITC-OVA) was efficiently internalized by DCs as early as 0.5 hrs' post-co-incubation in cell culture (FIG. 1A), indicating efficient cell uptake of particles. Transmission electron microscopy revealed that the PSM particles were surrounded by phagosome-like vesicular structures 3 hrs' post-incubation (FIG. 1B). Pre-treatment of DCs with either the phagocytosis inhibitor, cytochalasin D, or the macropinocytosis inhibitor, amiloride, significantly decreased particle internalization (FIG. 1C and FIG. 1D), suggesting that both phagocytosis and macropinocytosis were involved in the process. Interestingly, real-time particle tracking revealed that the DC-internalized particles could be transferred to the neighboring cells in vitro (FIG. 7D). When PSM-containing DCs were intravenously injected into mice, certain PSM particles parted from the injected cells, and likely phagocytosed by the endogenous cells (FIG. 7E).

T-cell response to antigen cross-presentation of PSM-loaded OVA (PSM/OVA) was next assessed in DCs using an in vitro antigen presentation assay. DCs pulsed with PSM/OVA induced significantly higher levels of IL-2 production in OVA-specific B3Z CD8 T cells than those pulsed with soluble OVA (FIG. 1E, FIG. 1F, and FIG. 7B), suggesting that cross-presentation of PSM/OVA was much more efficient than that of soluble OVA in DCs. Efficient cross-presentation of PSM/OVA was not PSM size-dependent, as DCs pulsed with OVA in different size PSM microparticles (diameters 0.6-2.5 μm) consistently induced higher levels of IL-2 production in B3Z cells than those pulsed with soluble OVA or liposomal OVA (FIG. 7C). Consequently, the 1-μm PSM particles were utilized in all further studies. It was subsequently determined whether PSM packaging could induce sustained antigen presentation. DCs were pulsed with either soluble or PSM-packaged OVA peptide for 2 hrs at 37° C., followed by extensive wash, and then either immediately co-cultured with B3Z cells or incubated in medium for 18 or 30 hrs before B3Z cell co-culture. IL-2 levels in the T-cell culture were comparable when DCs were used for co-culture immediately after removal of OVA peptide in either soluble or PSM formulation (FIG. 1G). However, T-cell activities were significantly higher in co-culture with PSM/peptide-pulsed DCs than soluble peptide-pulsed DCs 18 hrs and 30 hrs after peptide removal, indicating that PSM-delivered antigen induced prolonged MHC-peptide presentation to T-cells.

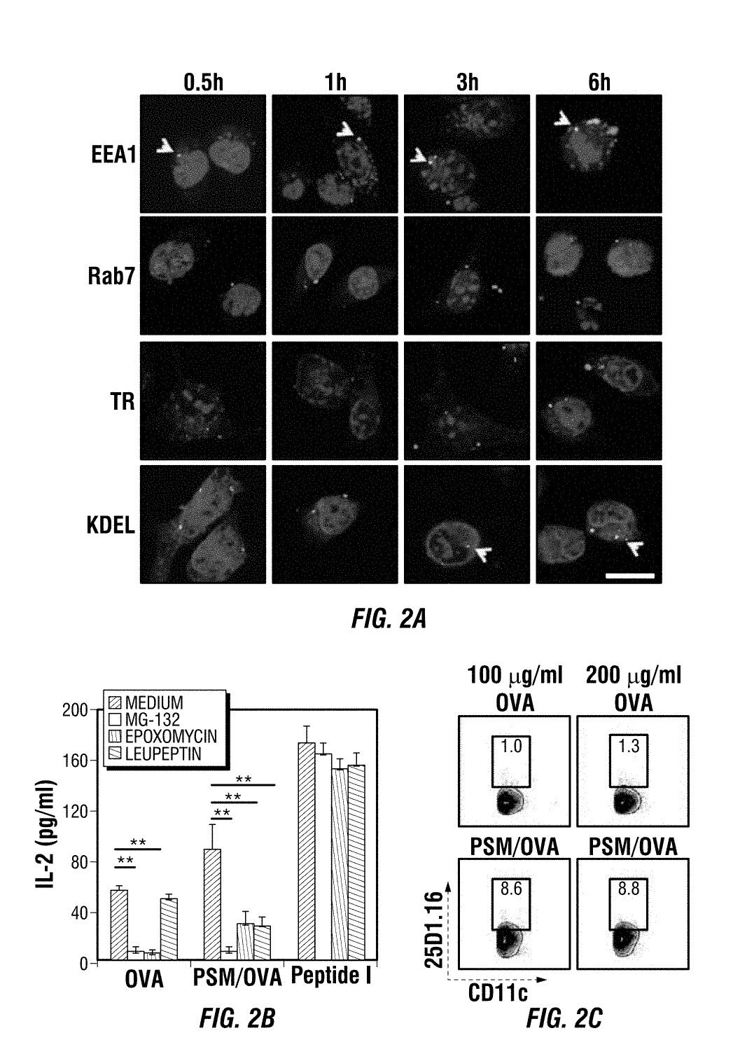

Antigen Presentation Pathway of PSM/OVA Antigen. Intracellular antigen trafficking was next examined using confocal microscopy. PSM/FITC-OVA particles co-localized with early endosomes (EEA1+) as early as 0.5 hr after co-incubation with DCs, and were in the endoplasmic reticulum (KDEL+) at 3 hrs' and 6 hrs' post-incubation. Particles were rarely found in late endosomes (Rab7+) or recycling endosomes (transferring receptor+) (FIG. 2A). Interestingly, PSM/FITC-OVA could still be spotted in early endosomes by 6 hrs, indicating that PSM/OVA could retain in this subcellular organelle for a prolonged time. In comparison, free FITC-OVA showed very weak signal by 6 hour, likely due to quick degradation inside the cells.

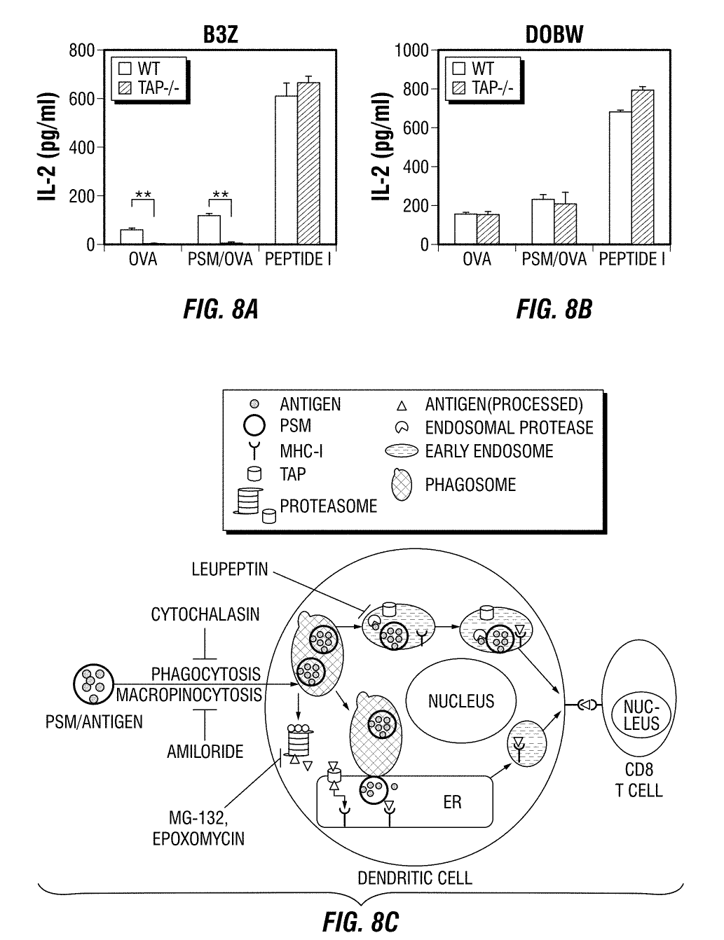

To identify route(s) of antigen processing of the PSM-packaged vaccine, DCs were treated with proteasome and lysosome inhibitors before co-incubation with soluble OVA or PSM/OVA, and measured induction of IL-2 expression. Proteasome inhibitors (MG-132 and epoxomycin), but not the lysosome inhibitor leupeptin, completely blocked class I antigen presentation of soluble OVA (FIG. 2B). In contrast, PSM/OVA-induced IL-2 production was inhibited by all inhibitors, suggesting that both the proteasome and lysosome pathways were involved in antigen presentation of PSM/OVA. Since the transporter associated with the antigen processing (TAP) protein is an essential component of class I antigen presentation pathway (Trombetta and Mellman, 2005), antigen processing was tested in DCs isolated from TAP1 knockout mice (TAP^{-/-} DCs). As expected, class I antigen presentation of PSM/OVA was abolished in TAP^{-/-} DCs (FIG. 8A). Conversely, class II antigen presentation of PSM/OVA was similar in both WT and TAP^{-/-} cells (FIG. 8B). Thus, PSM/OVA cross-presentation by DCs requires TAP-dependent antigen processing.

Since OVA antigen processing resulted in surface expression of OVA peptide bound class I MHC molecules, the processed antigen was probed with antibody 25D1.16, which specifically recognizes the MHCI-peptide (SIINFEKL-H-2K^{b}) complex. Indeed, more 25D1.16-positive cells could be identified in PSM/OVA-primed DCs than in soluble OVA-primed DCs (FIG. 2C), thus confirming enhanced antigen processing, and presentation of PSM loaded OVA by DCs.

These observations indicated that, after internalization by DCs through phagocytosis/macropinocytosis, PSM/OVA leaves the phagosome, and the released antigen is processed through the proteasome pathway. The processed antigen binds to MHC molecules in the ER, and is then transported to the cell surface. Alternatively, the antigen can be processed directly by endosomal proteases in early endosomes, and presented to the cell surface in the form of an antigen peptide-MHC complex. In either case, the TAP protein was required for loading antigen peptide onto MHC molecules (FIG. 8C).

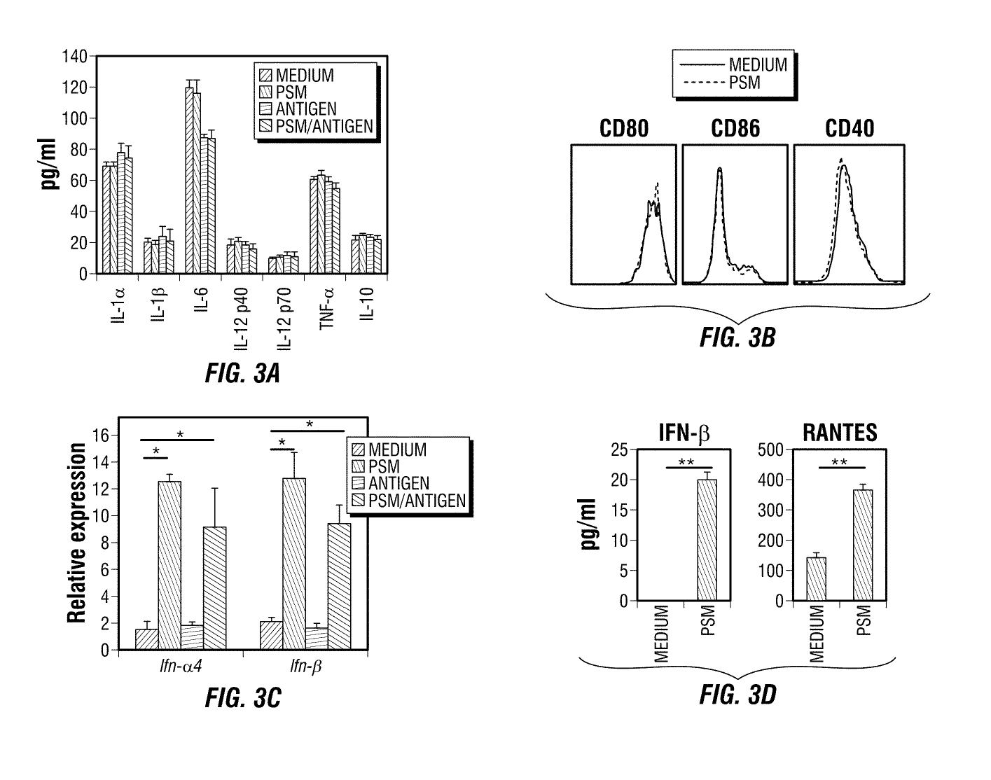

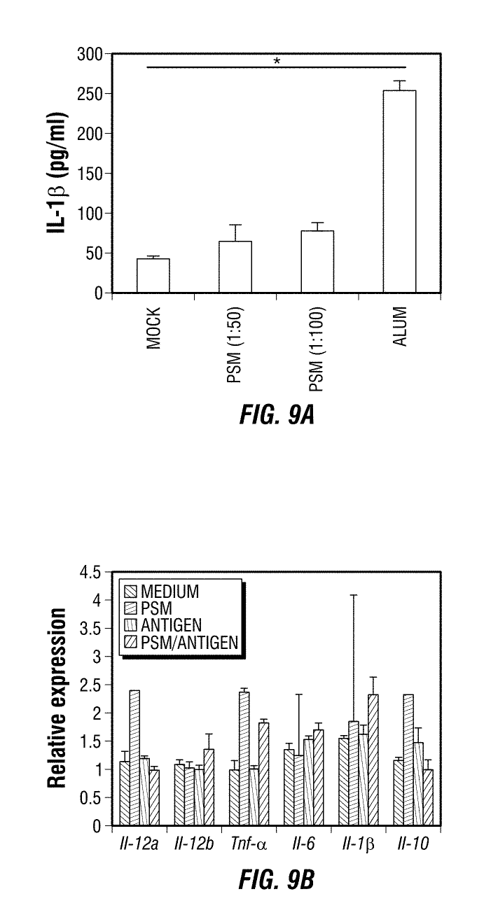

PSM-Induced Innate Immune Responses. It was reasoned that the enhanced cross-presentation by PSM loaded antigen might be associated with activation of innate immunity in addition to early endosome delivery. While alum adjuvant can activate inflammasomes and induce IL-1β maturation and release in DCs, PSM did not induce IL-1β release in LPS-primed DCs (FIG. 9A). Neither PSM, nor antigen-loaded PSM altered either the mRNA or protein levels of pro-inflammatory cytokines (see FIG. 3A and FIG. 9B). Furthermore, there was almost no change in expression of surface co-stimulatory molecules (CD80, CD86, and CD40) after PSM treatment (see FIG. 3B).

As one important arm of innate immune responses to viral infection, IFN-I response is required for optimal T-cell activation by promoting cross-presentation and stimulating CD8 T-cell clonal expansion (Le Bon and Tough, 2008; Ng and Gommerman, 2013). Expression profiles of IFN-I-related genes were analyzed, and it was found that both empty PSM and antigen-loaded PSM (but not free antigen) induced a significant increase in IFN-α4 and IFN-β expression (FIG. 3C). ELISA results confirmed that PSM-treated DCs exhibited a modest but significant increase in IFN-β production, and also secreted a high level of RANTES, an IFN-I regulated chemokine (FIG. 3D).

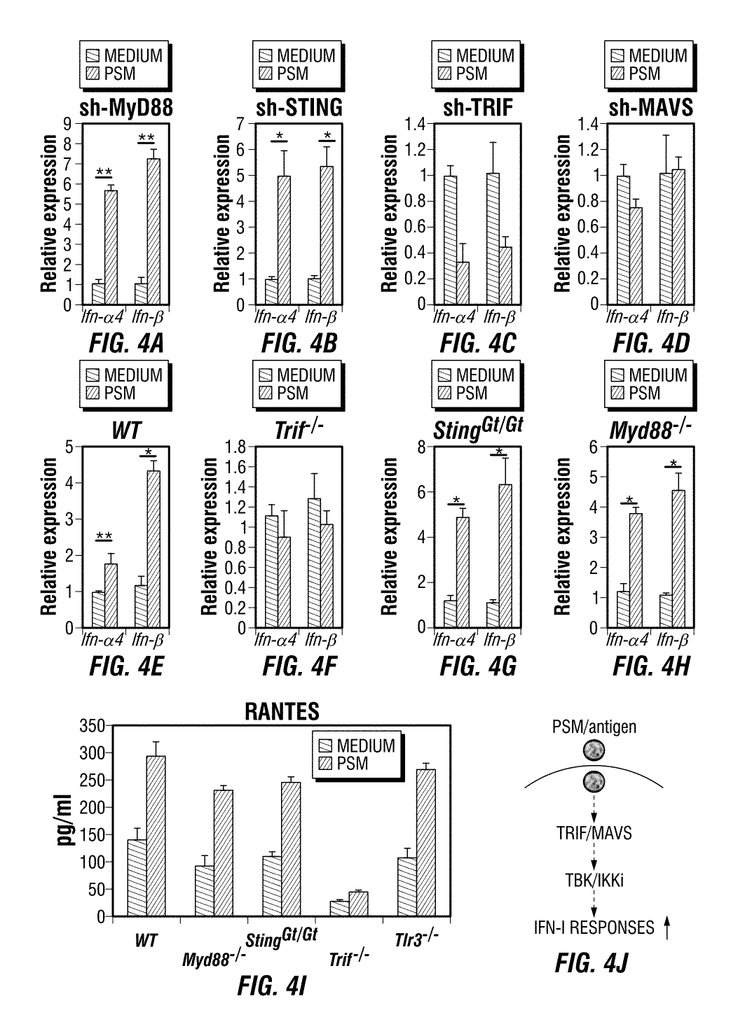

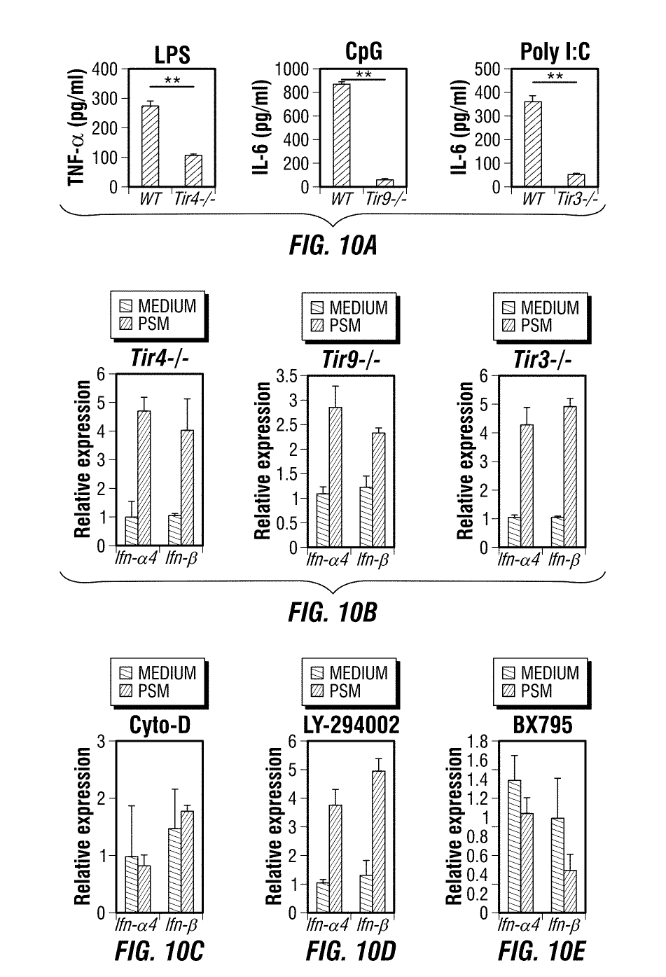

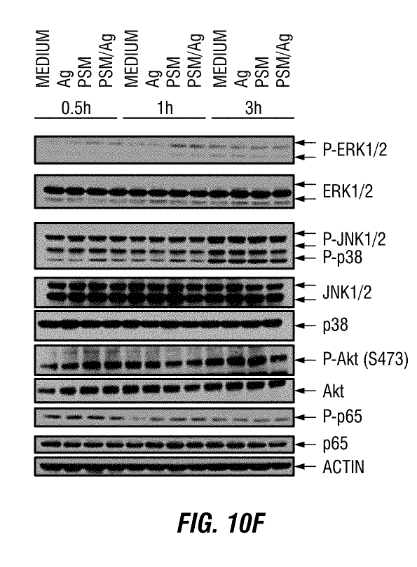

PSM-Induced IFN-I Response is Dependent on TRIF and MAVS Signaling. To determine whether the IFN-I responses elicited by PSM was dependent on any known TLR- or RLR-induced IFN-I pathways, expression of key adaptor molecules in these pathways was knocked down, and then PSM-induced IFN-I responses was analyzed in DCs (FIG. 4A, FIG. 4B, FIG. 4C, and FIG. 4D). PSM retained the ability to induce IFN-I response with suppressed expression of MyD88 and STING. In contrast, IFN-I induction was abolished when TRIF or MAVS was knocked down, suggesting that PSM induced IFN-I response through TRIF- and MAVS-dependent pathways. Bone marrow-derived dendritic cells (BMDCs) from gene knockout mice were then applied to validate the results from shRNA-based studies. IFN-I induction by PSM was completely inhibited in Trif−/− BMDCs, but not in Sting^{Gt/Gt} (Sauer et al., 2011) or Myd88−/− BMDCs (FIG. 4E, FIG. 4F, FIG. 4G, and FIG. 4H). Consistently, PSM treatment enhanced RANTES production in BMDCs from WT, Myd88−/−, Sting^{Gt/Gt} and Tlr3^{-/-} mice; however, RANTES production was inhibited in Trif−/− BMDCs (FIG. 4I). Although TLR4, TLR9, and TLR3 deficiency abolished DC response to LPS, CpG, and polyI:C respectively (FIG. 10A), PSM-induced IFN-I response was not affected (FIG. 10B), suggesting a TLR-independent mechanism for PSM-induced IFN-I responses.

Since PSM uptake and cross-presentation requires phagocytosis of PSM/antigen, the next study examined whether phagocytosis is required for PSM-induced IFN-I. Pre-treatment with the phagocytosis inhibitor, cytochalasin D, suppressed IFN-I induction by PSM (FIG. 10C). Downstream kinase signaling of phagocytosis was also analyzed. PI3K inhibition had no effect on IFN-I induction (FIG. 10D). In comparison, inhibition of the key kinases (TBK1/IKKi) for IFN-I signaling by BX795 markedly inhibited IFN-I induction (FIG. 10E). Activation status of MAPKs and NF-κB signaling in BMDCs after PSM/antigen priming was further analyzed. Except for a slight increase in the level of ERK phosphorylation, PSM priming did not strongly activate signaling of JNK, p38, AKT, or NF-κB (FIG. 10F). Taken together, these data indicated that phagocytosis of PSM-loaded antigens by DCs activated TRIF- and MAVS-dependent signaling and the downstream TBK1/IKKi to elicit IFN-I responses (FIG. 4J).

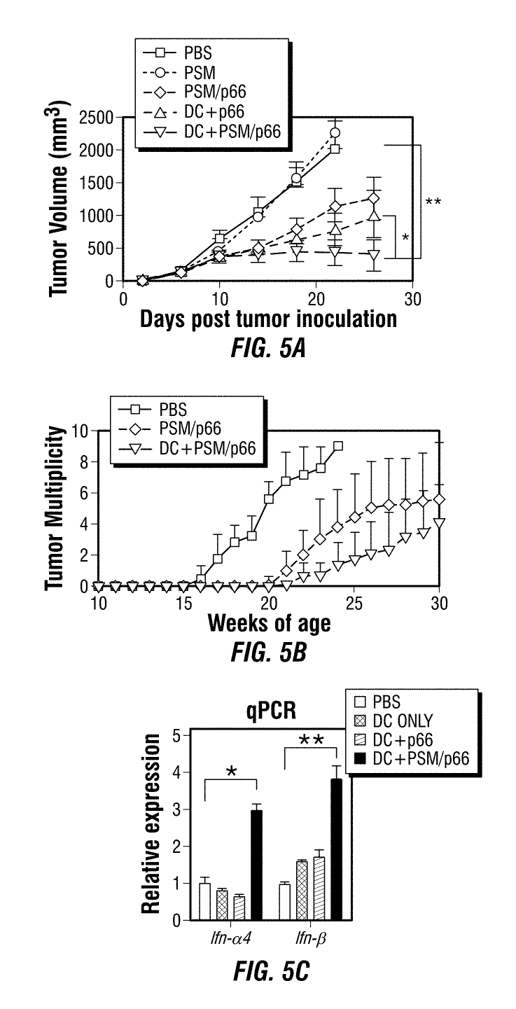

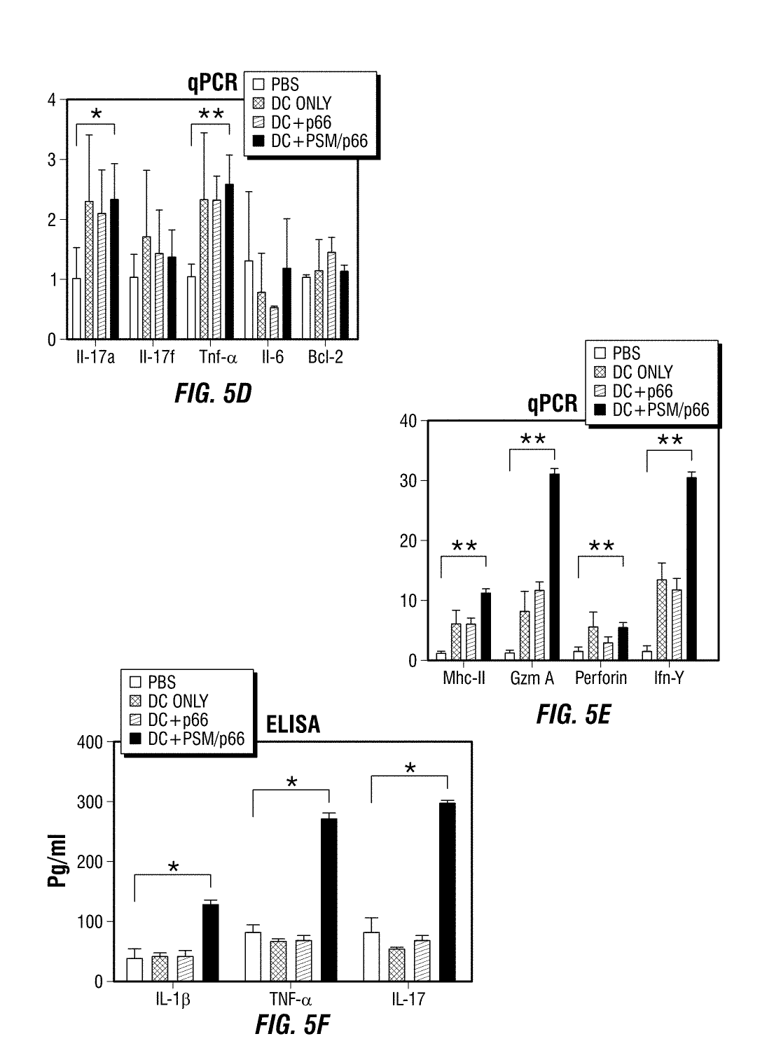

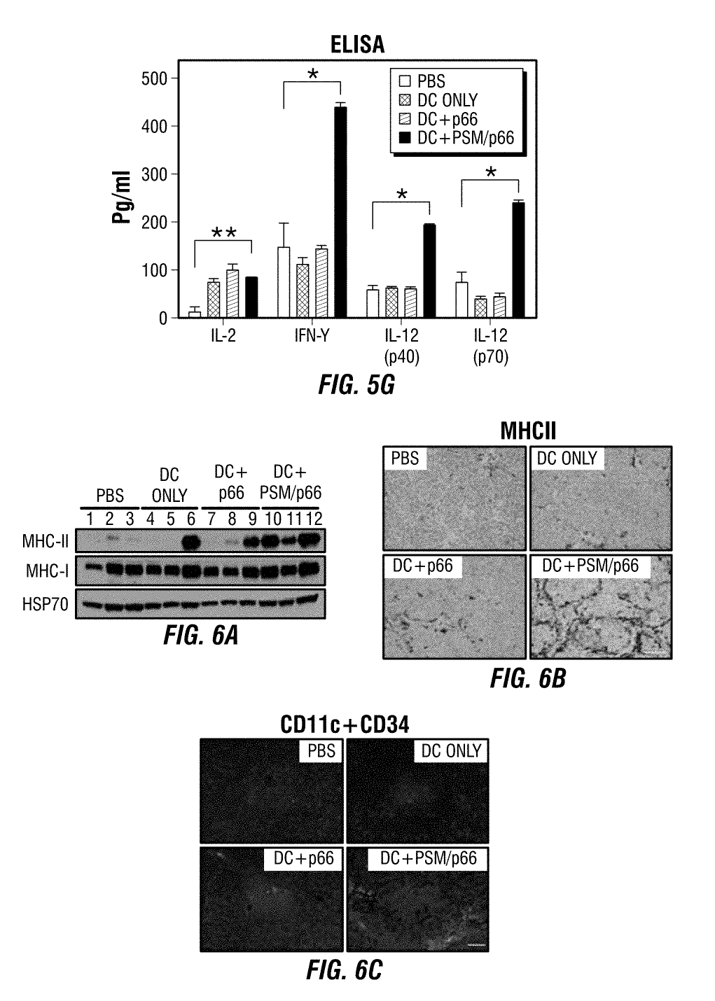

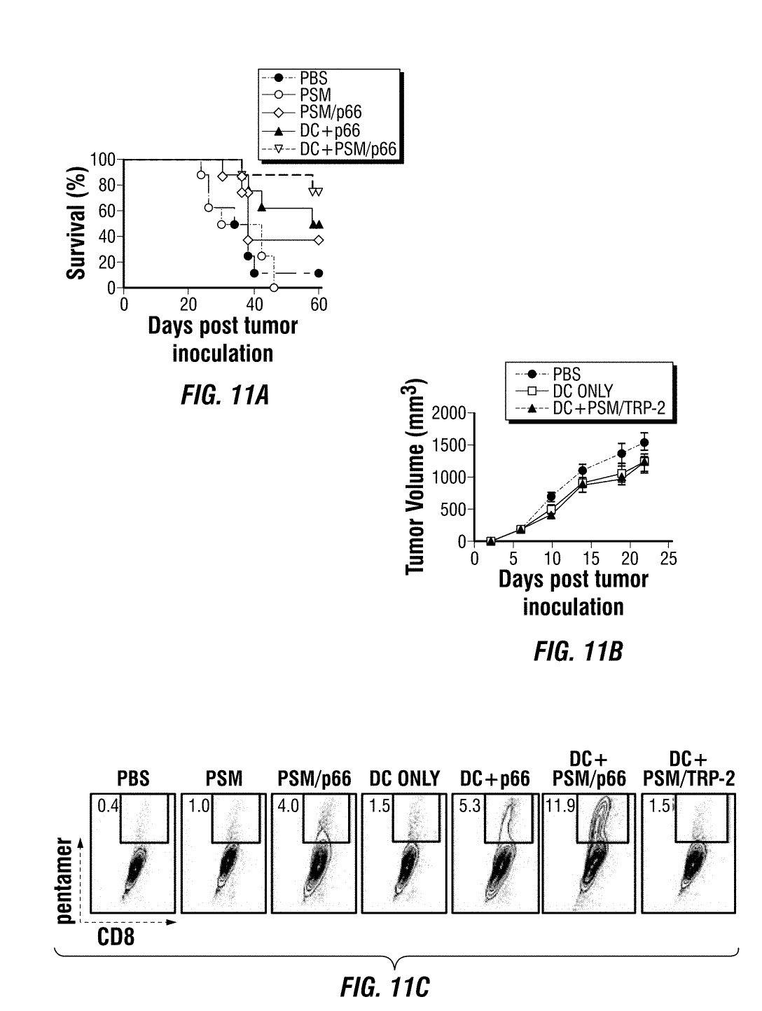

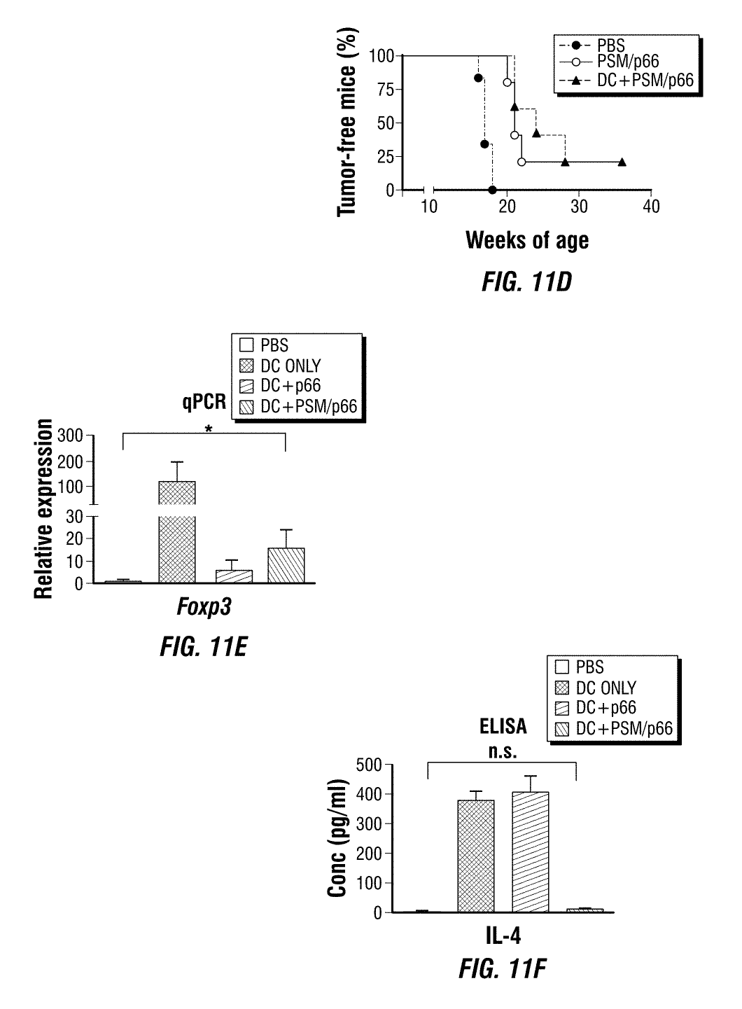

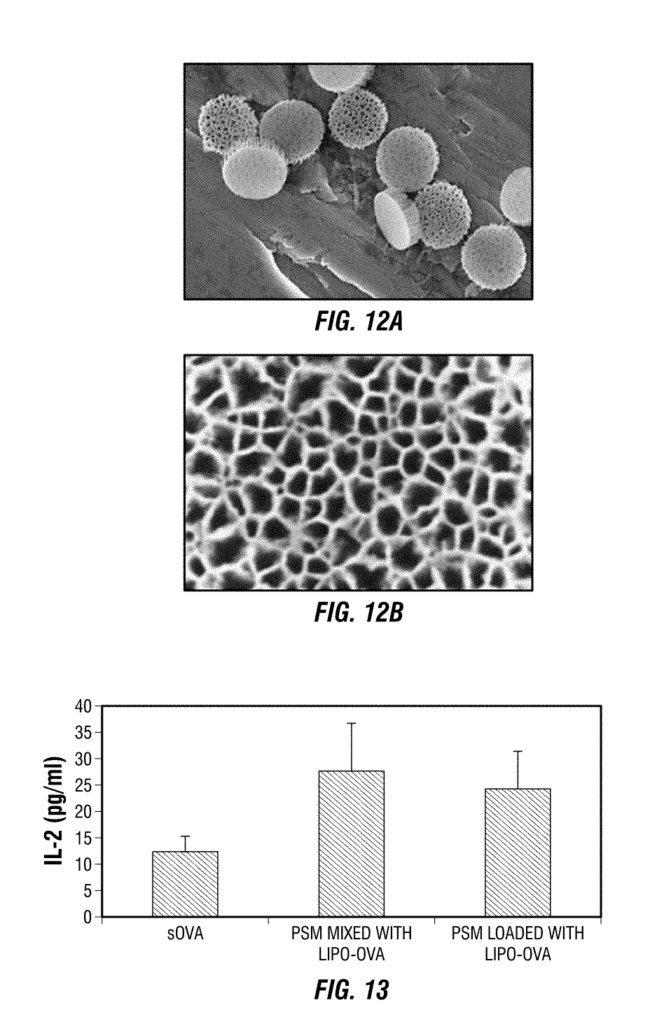

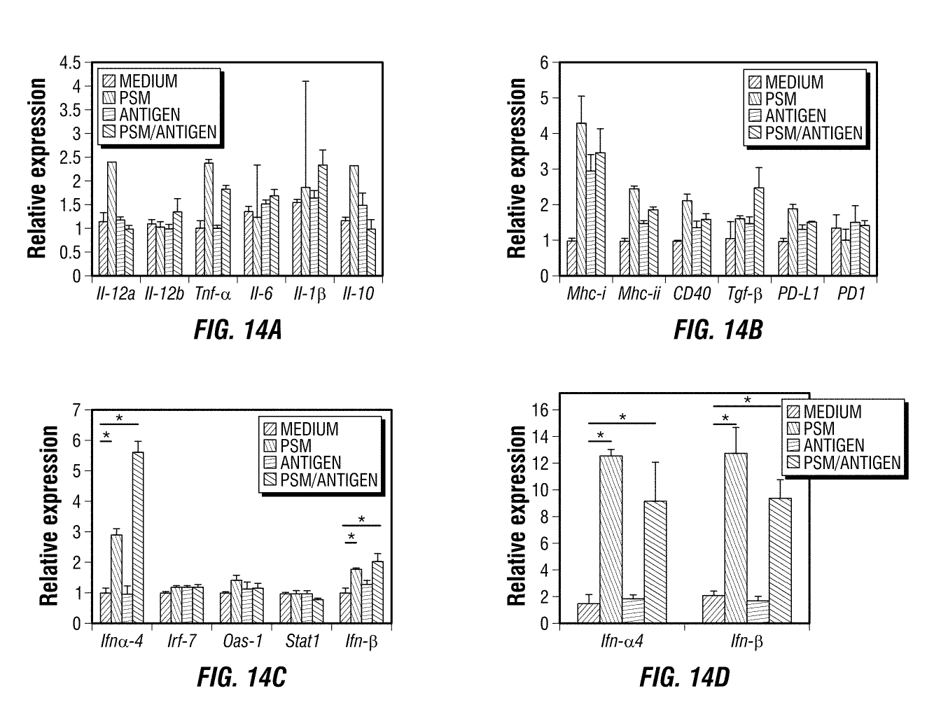

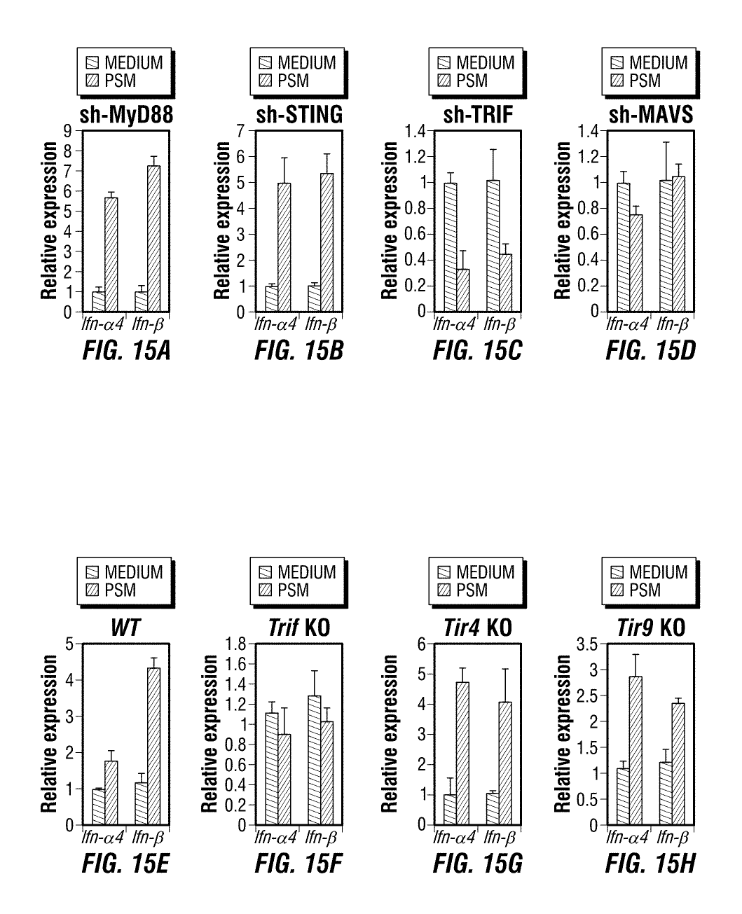

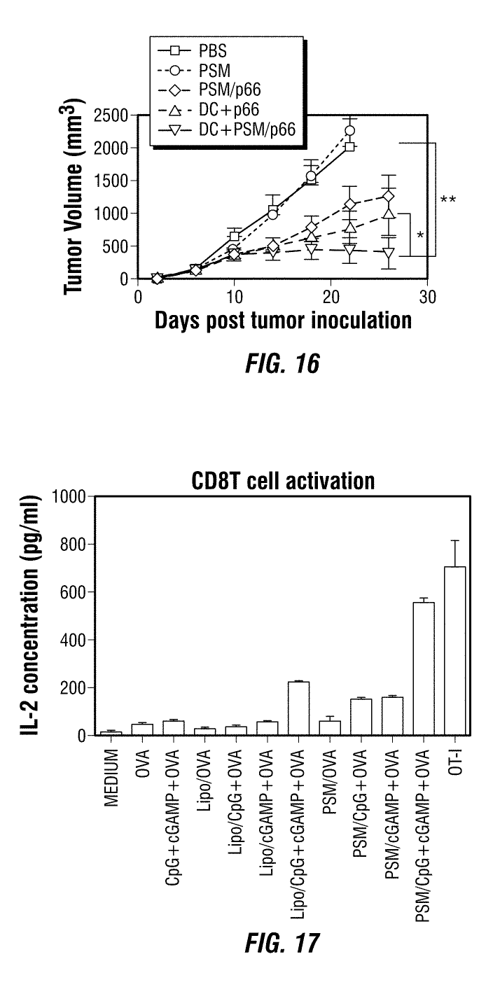

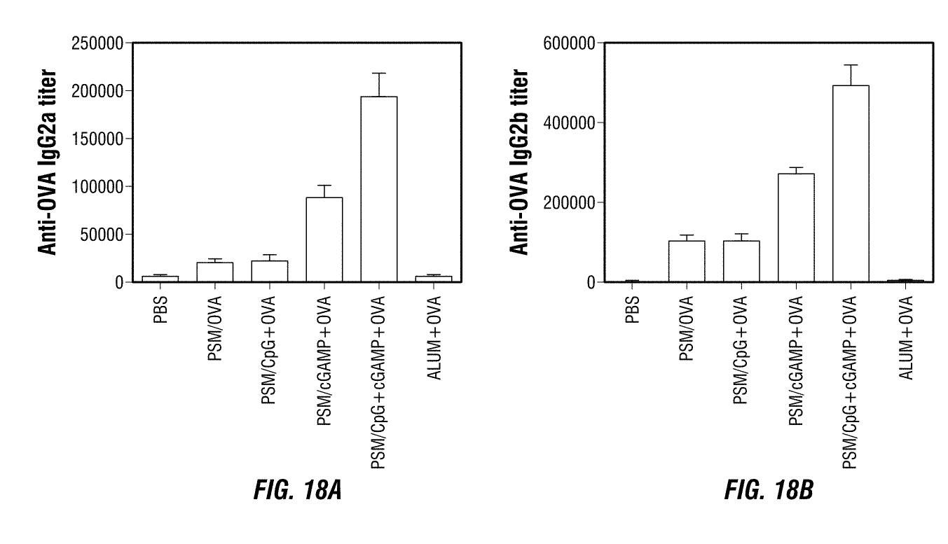

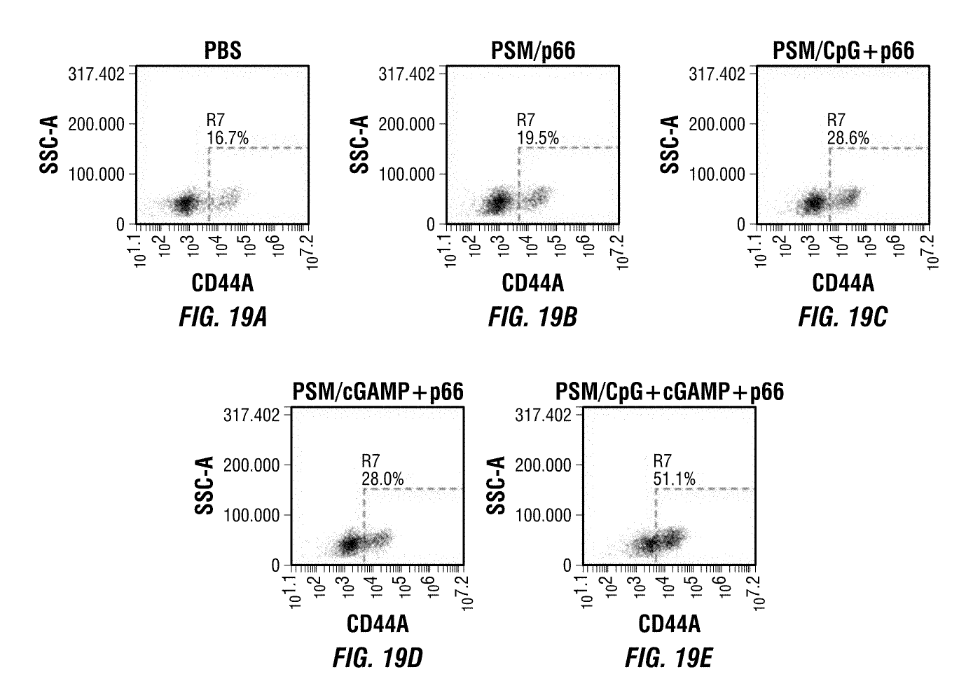

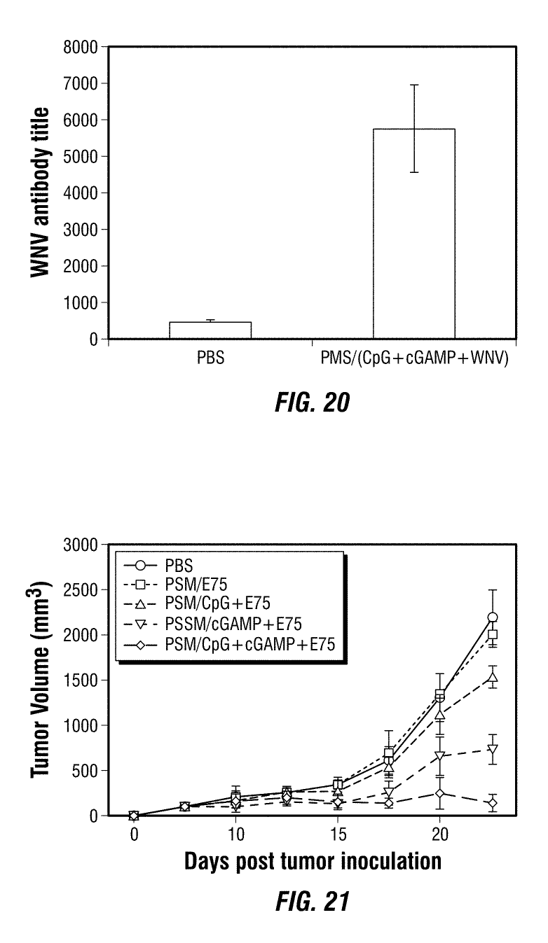

Anti-Tumor Efficacy of DCs Primed with PSM/HER2 Antigen Peptide. Given the critical role of efficient cross-presentation and CD8 T cell cytotoxicity in anti-tumor immunity, a further study examined whether enhanced cross presentation and activated IFN-I in DCs by PSM/antigen could be translated into enhanced anti-tumor activity in animal tumor models. BALB/c mice were inoculated with TUBO tumor cells, which were originally derived from mammary gland tumor of BALB-neuT transgenic mice (overexpressing rat neu under MMTV promoter) (Lucchini et al., 1992). When tumors reached a palpable size, the mice were treated i.v. with PSM, PSM/p66 (p66 is a class I HER2 antigen peptide) (Nagata et al., 1997), p66-primed DCs (DC+p66), or PSM/p66-primed DCs (DC+PSM/p66). While PSMs had no impact on tumor growth, direct PSM/p66 administration inhibited tumor growth significantly (FIG. 5A). Mice that received DC+p66 showed comparable tumor growth inhibition as those in the PSM/p66 treatment group. The most significant effect was observed in mice treated with DC+PSM/p66, demonstrated by the nearly complete tumor growth inhibition and extended animal survival (FIG. 5A and FIG. 11A). The effect on tumor growth was specific for HER2 peptide p66, as treatment with DC only or DCs primed with PSM-TRP-2 (a non-related antigen) could not inhibit tumor growth (FIG. 11B). In addition, the anti-tumor efficacy correlated with intratumor HER2-specific CD8 T cell frequency (FIG. 11C).

In a separate study, the tumor prevention effect from PSM/p66 was evaluated. BALB-neuT mice started to develop spontaneous tumors at 15 weeks, and all mice developed tumors by week 17 (FIG. 11D). There were on average over five tumors per mouse by week 20 (FIG. 5B). Mice treated with PSM/p66 or DC+PSM/p66 developed significantly less tumors and exhibited longer tumor latency than the non-vaccinated mice (FIG. 5B and FIG. 11D). The results indicated that PSM-loaded HER2 peptide served as an anti-HER2 vaccine and prevented mammary gland tumor development in the transgenic mice.

PSM/Antigen-Primed DC Elicited Immunostimulating Tumor Micro-Environment Intratumor cytokine levels were quantitated in post-treatment TUBO tumors, since anti-tumor immunity is often dependent on the immune-stimulating tumor microenvironment (Coussens et al., 2013). Consistent with the cell-based study (FIG. 3C), quantitative RT-PCR (qPCR) analysis revealed significantly increased intratumor IFN-I levels in vaccinated mice (FIG. 5C). Levels of pro-inflammatory cytokines were mixed. While expression of TNF-α and IL-17a increased in the vaccinated mice, IL-17F, IL-6 and the anti-apoptotic molecule Bcl-2 remained unchanged (FIG. 5D). On the other hand, expression of the antigen presentation molecule MHC-II significantly increased in vaccinated groups (FIG. 5E). Moreover, levels of cytotoxic T cell markers (granzyme and perforin) and IFN-γ also increased after vaccination, with granzyme and IFN-γ levels being 2-3 fold as high in the DC+PSM/p66 treatment group as in the DC or DC+p66 treatment groups (FIG. 5E). Interestingly, all DC vaccines induced intratumoral expression of the Treg marker gene Foxp3 (FIG. 11E), suggesting vaccination induced Treg expansion and tolerance (Ambrosino et al., 2006). To confirm the qPCR results, single cells were isolated from tumor samples, grown in culture, and cytokine levels were measured in the cell growth medium by ELISA. Significantly increased levels of key pro-inflammatory cytokines such as IL-1β, IL-17 and TNF-α were detected in tumor cells from the DC+PSM/p66 treatment group compared to the other groups (FIG. 5F). In addition, Th1 cytokines such as IFN-γ, IL-12p40 and IL-12p70 were the highest in the DC+PSM/p66 treatment group (FIG. 5G). As BALB/c mice are prone to Th2 response (Mills et al., 2000), a dramatic increase in level of the Th2 cytokine IL-4 was observed after DC and DC+p66 treatment (FIG. 11F). However, DC+PSM/p66 treatment did not induce IL-4 production. Thus, PSM/p66 priming could obviate the strong Th2 response associated with DC treatment in BALB/c mice. These results suggest that DC+PSM/p66 induced a pro-inflammatory environment in tumor tissues, and shifted an otherwise predominant Th2 response in BALB/c mice to a Th1 response, which may contribute to the anti-tumor immunity.

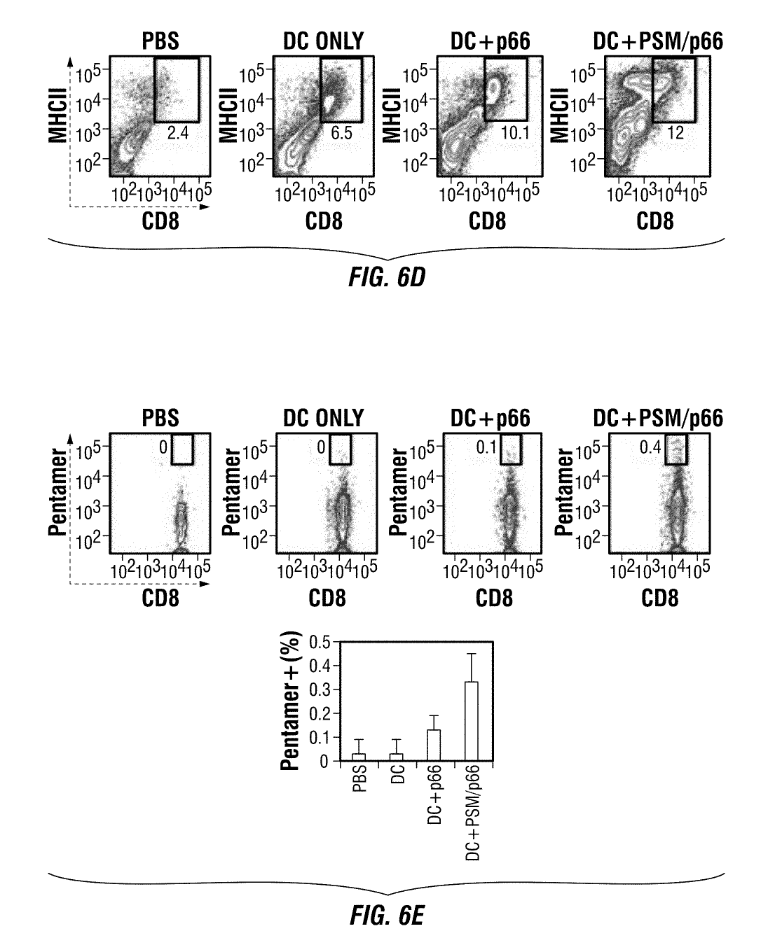

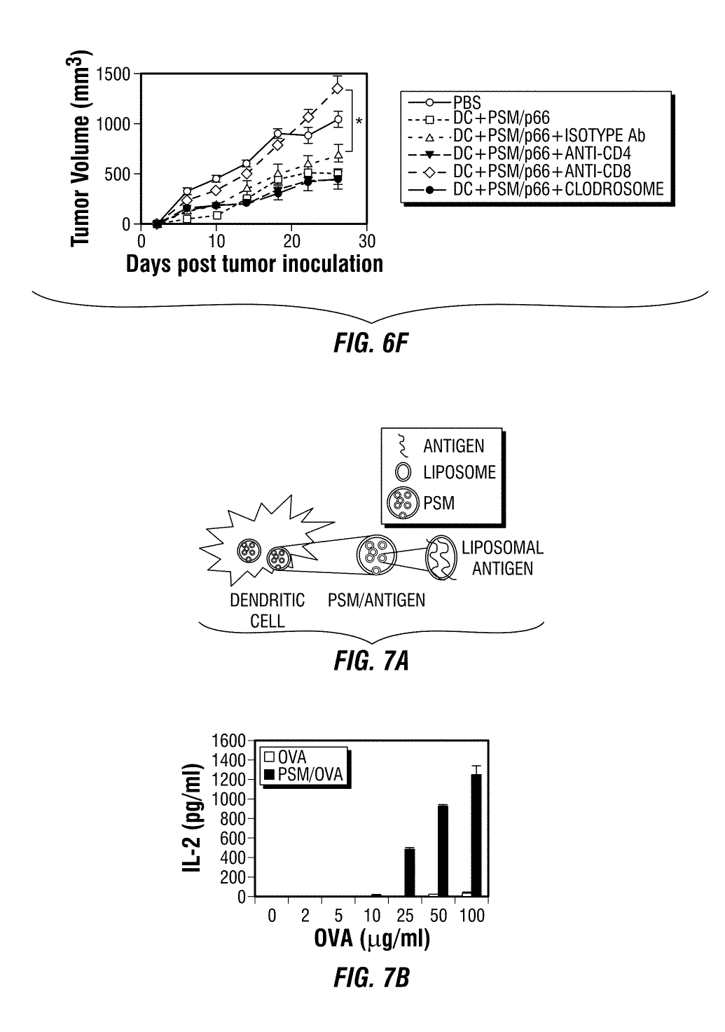

PSM/HER2-Primed DC Vaccine Activated Cytotoxic T-Cell Immunity Against Tumor. Intratumor cell populations that might contribute to the intratumor cytokine milieu were further analyzed. Western blot analysis of tumor tissue lysates revealed comparable MHC-I protein levels among the treatment groups; however, MHC-II expression dramatically increased in tumor from the DC+PSM/p66 group (FIG. 6A). Immunohistochemical (IHC) staining confirmed a high level of MHCII positive cells on the edge of tumor nodules in the DC+PSM/p66 group (FIG. 6B). Immunofluorescent staining and flow cytometry analysis revealed markedly increased percentage of CD11c+MHCII+cells in tumors from the DC+PSM/p66 group over the other groups (FIG. 6C and FIG. 6D).

As the executor of T cell-mediated anti-tumor immunity, HER2-specific CD8 T cells had the highest level in the DC+PSM/p66 treated tumors (FIG. 6E). To determine which type(s) of immune cells were required for the anti-tumor immunity, CD4 T cells, CD8 T cells, or macrophages were depleted in mice bearing TUBO tumors, and then mice were treated with DC+PSM/p66 (FIG. 6F). CD8 T-cell depletion completely abolished anti-tumor immunity. In contrast, depletion of CD4 T cells or macrophages did not impair the anti-tumor immunity; neither did treatment with an isotype control antibody. These results indicated that CD8 T cells were essential for DC+PSM/p66-induced anti-tumor activity.

FIG. 12A and FIG. 12B show scanning electron microscopy (SEM) images of exemplary 1 μm×0.4 μm discoidal microparticles in accordance with one aspect of the present invention. PSM can be fabricated in a variety of different sizes and shapes. Discoidal (i.e., platelet-like), hemispherical, cylindrical, and other types of pSi particles have been developed. A variety of discoidal pSi particles have been fabricated, ranging in size from about 1.0 μm to about 3.5 μm in diameter, and from about 0.2 μm to about 0.7 μm in thickness.

The data presented in FIG. 13 showed that in order to determine whether PSM could serve as an adjuvant to boost vaccination, ovalbumin (OVA), a test antigen, was mixed with either PSM, or loaded with OVA into the nanopores of PSM, and then incubated with dendritic cells (DCs) for three hrs. Afterwards, the DCs were co-incubated for 14-18 hrs with B3Z CD8 T cells, which recognized the OVA antigen peptides. CD8 T cells secreted interleukin 2 (IL-2) into culture medium, indicating the cells had been activated. Comparing cells incubated with ovalbumin only (negative control, without PSM), DC incubated with either a simple mix of PSM+ovalbumin or ovalbumin-loaded PSM significantly stimulated IL-2 expression. There was no difference on IL-2 secretion from CD8 T cells co-incubated with dendritic cells with either pre-treatment. This result demonstrated that PSM promoted the processing of ovalbumin and antigen peptide presentation by the dendritic cells, and that a mixture containing only PSM+antigen was sufficient to enhance the performance of dendritic cells.

The data presented herein demonstrates that the disclosed PSMs can be utilized as potent adjuvants for cancer vaccines, and that this activity results via a previously undescribed mechanism of action (i.e., by the induction of a Type-I interferon response). In contrast to existing adjuvants such as nanosized alum crystals, which activate inflammasomes, and promote IL-1β release in dendritic cells, the PSM compositions disclosed herein function as vaccine adjuvants by triggering a type I interferon-mediated cellular response. Compared to conventional particulate adjuvants of the prior art, the present adjuvant compositions specifically enhance antigen presentation, and avoid many of the broad untoward consequences of systemic inflammation.

It has been suggested that phagocytosis of particulate antigens or adjuvants not only facilitates their uptake by APCs, but also triggers innate immune activation by interaction with innate immune receptors (Fric et al., 2014). In this example, it has been demonstrated that phagocytosis was required both for cross-presentation and IFN-I induction in PSM/antigen-primed DCs, reinforcing the dual role of phagocytosis in antigen uptake and innate immune activation.

IFN-I response is elicited in DCs after exposure to microbial or tumor-derived DNA and RNA. Binding of nucleotides activates innate immune receptors such as TLRs and RLRs, and subsequently recruits individual adaptor proteins and the key kinases TBK1 and IKKi, leading to IRF3 or IRF7 phosphorylation, nuclear translocation and activation of IFN-I gene transcription (Prinz and Knobeloch, 2012). It is generally accepted that MyD88 is required for TLR7/8/9-induced IFN-I signaling, while TRIF is needed for TLR3/4-induced IFN-I signaling. The intracellular RNA and DNA sensors rely on adaptor molecules MAVS and STING for signaling transduction (Prinz and Knobeloch, 2012). Interestingly, membrane fusion can directly activate IFN-I response through STING-dependent signaling without activating DNA or RNA sensors, suggesting that physical fusion sometimes serves as “danger signal” to elicit innate immune response (Holm et al., 2012). However, membrane fusion is unlikely the primary factor for PSM-induced IFN-I response, as knockdown or knockout of STING in DCs did not abolish PSM-induced IFN-I responses. It is intriguing that both TRIF and MAVS are essential for PSM-induced IFN-I response, since they seem to mediate different upstream signals from TLRs and RLRs. However, MAVS was initially identified as a downstream adaptor of TRIF in intracellular poly(I:C) induced IFN-I signaling (Xu et al., 2005), and both TRIF and MAVS are required for DDX1/DDX21/DHX36 complex-induced IFN-I activation (Zhang et al., 2011). Therefore, a TRIF-MAVS axis may exist for IFN-I induction in response to intracellular DNA/RNA molecules, or to phagocytosed PSM particles. It is noteworthy that PSM induced moderate IFN-I expression. Such a level of IFN-I is competent enough for stimulating cross-presentation and CD8 T cell expansion, as demonstrated in lymphotoxin receptor-mediated IFN-I induction (Summers deLuca et al., 2011).

In the context of the tumor microenvironment, although endogenous immunity against cancer still exists, many aspects of anti-tumor immunity have been crippled during tumor development, and thus require exogenous boosting treatment (Gajewski et al., 2013a). Increased levels of IFN-I have been reported as favorably correlating with clinical immune responses against cancer (Fuertes et al., 2011), and intratumoral IFN-I levels predict clinical responses of breast cancer patients to anthracycline-based chemotherapy (Sistigu et al., 2014). Exogenous therapeutic antibody coupled with IFN-β also enhances anti-tumor activity by activating the cross-presentation capacity of endogenous dendritic cells (Yang et al., 2014). Therefore, targeting DCs and re-activating their cross-presentation capacity represent an important strategy for cancer immunotherapy.

Dendritic cell-based immunotherapy can be performed by administration of exogenously prepared DCs, or by injection of antigens and adjuvants that target endogenous DCs. Mechanistically, injected DCs may present antigen directly to T-cells, or alternatively, may serve as a cellular delivery vector and transfer the antigen to endogenous DCs (Kleindienst and Brocker, 2003; Yewdall et al., 2010).

While not being bound by any particular theory, it is tempting to speculate that the PSM/antigen-loaded DCs may transfer PSM/antigen to endogenous DCs and promote their anti-tumor immunity. Data presented in this example support such a scenario, as inter-cellular exchange of PSMs was observed between DCs in vitro, and PSMs released from injected DCs in vivo.

Previous reports have also identified anti-tumor immunity accompanied with increased intratumoral MHC-II expression after treatment with the agonist anti-CD40 antibody, which was attributed to activated innate immune cells, mainly macrophages (Beatty et al., 2011; O'Sullivan et al., 2012). In this example, PSM/antigen-primed DC treatment also drastically increased MHC-II expression in tumor tissues. Nevertheless, depletion of macrophages in the TUBO tumor model did not have a significant impact on DC-based therapeutic effect. Instead, increased tumor infiltration of CD11c+ cells accompanied with MHC-II upregulation was observed in mice treated with PSM/HER2 primed DCs, demonstrating the critical role of activated DCs in anti-tumor activity. These findings were consistent with a recent report describing activation of intratumoral dendritic cells by IFN-I, and enhanced antibody-mediated tumor immunity (Yang et al., 2014).

CD8 T cells are regarded as the key player in anti-tumor activity (Dougan and Dranoff, 2009). It has been shown that depletion of CD8 T cells abrogates anti-tumor activity of PSM/antigen primed DC vaccine. This result was consistent with previous reports using the same CD8 epitope HER2 peptide that anti-tumor immunity is mainly dependent on CD8 T cell cytotoxic killing activity (Assudani et al., 2008; Nava-Parada et al., 2007). Another important observation is that vaccination also induced FOXP3 expression in tumor tissues. Treg infiltration is a major barrier for inducing effective anti-tumor immune responses both in human clinical trials (Gajewski et al., 2013b) and in murine tumor models such as the BALB-neuT transgenic mice (Ambrosino et al., 2006). Transient Treg ablation by antibody or genetic manipulation has shown antitumor effect by itself (Bos et al., 2013; Marabelle et al., 2013). This may explain why CD4 antibody depletion did not affect anti-tumor immunity of DCs primed with PSM/antigen, as CD4 antibody may also deplete Tregs in addition to T helper (T_{H}) cells. Based on these results, a combinational treatment with PSM/antigen-primed DC vaccine and transient Treg ablation may further improve the outcome of anti-tumor vaccines.

PSM/antigen-primed DC vaccination was shown to produce a strong anti-tumor immunity by inducing efficient antigen cross-presentation and eliciting IFN-I response. Phagocytosis of PSM/antigen generated prolonged early endosome antigen localization and simultaneously activated IFN-I signaling, thus providing another example of how phagocytosis and innate immune signaling cooperate to enhance cross-presentation. A specific intracellular pathway that mediates PSM-induced IFN-I signaling has been identified, illustrating the essential functions of TRIF and MAVS in response to particulate antigens. In addition, the role of immunocompetent tumor microenvironment has been demonstrated in PSM-potentiated DC vaccines. These findings further highlight the advantage of particulate vaccines, and IFN-I signaling in stimulating the tumor immune microenvironment for successful immunotherapy.

### Example 2

### PSM Adjuvants Enhance Antigen Cross-Presentation & Induce IFN-I Response

The present example demonstrates that PSM could synergize with other adjuvants to stimulate dendritic cells and consequently activate the CD8-positive cytotoxic T cells (essential for therapeutic cancer vaccine) and CD4-positive T help cells (essential for T cell and B cell activation). The results indicated that PSM can serve as a potent adjuvant not only for therapeutic cancer vaccine (CD8 T cell activity), but also for vaccines to target infectious diseases (CD4 T cell activity).

These studies demonstrated that 1) PSM synergized with other adjuvants to boost vaccination efficiency, 2) PSM was potent in stimulating antibody production (CD4+ T cell activity), and 3) PSM boosted memory T cell population.

### Example 3

### PSM Adjuvants Promote Antibody Production Against Infectious Diseases

The present Example demonstrates that PSM-based adjuvants were effective in treating infectious diseases.

To illustrate the efficacy of the disclosed PSM-based adjuvants in treating infectious diseases, a West Nile Virus (WNV) protein was mixed with PSM+CpG+cGAMP, and then wild-type mice were injected with either phosphate buffer saline control or PSM(CpG+cGAMP+WNV). Blood samples were then collected at 2, 3, and 4 weeks' post-injection, and levels of WNV-specific antibody were quantitated. FIG. 17 illustrates the result from the first sample set (2 weeks after vaccination).

### REFERENCES

The following references, to the extent that they provide exemplary procedural or other details supplementary to those set forth herein, are specifically incorporated herein in their entirety by express reference thereto:

AMBROSINO, E et al., “Immunosurveillance of Erbb2 carcinogenesis in transgenic mice is concealed by a dominant regulatory T-cell self-tolerance,” Cancer Res., 66(15):7734-7740 (2006).

ASSUDANI, D et al., “In vivo expansion, persistence, and function of peptide vaccine-induced CD8 T cells occur independently of CD4 T cells,” Cancer Res., 68(23):9892-9899 (2008).

AUDIBERT, F, “Adjuvants for vaccines, a quest,” Int. Immunopharmacol., 3(8):1187-1193 (2003).

BACHMANN, M F, and JENNINGS, G T, “Vaccine delivery: a matter of size, geometry, kinetics and molecular patterns,” Nat. Rev. Immunol., 10(11):787-796 (2010).

BARNIER-QUER, C et al., “Adjuvant effect of cationic liposomes for subunit influenza vaccine: influence of antigen loading method, cholesterol and immune modulators,” Pharmaceutics, 5(3):392-410 (2013).

BEATTY, G L et al., “CD40 agonists alter tumor stroma and show efficacy against pancreatic carcinoma in mice and humans,” Science, 331(6024):1612-1616 (2011).

BOS, P D et al., “Transient regulatory T cell ablation deters oncogene-driven breast cancer and enhances radiotherapy,” J. Exp. Med., 210(11):2435-2466 (2013).

BRAUMULLER, H et al., “T-helper-1-cell cytokines drive cancer into senescence,” Nature, 494(7437):361-365 (2013).

BREWER, J M, “(How) do aluminium adjuvants work? Immunol. Lett., 102(1):10-15 (2006).

BURGDORF, S et al., “Spatial and mechanistic separation of cross-presentation and endogenous antigen presentation,” Nat. Immunol., 9(5):558-566 (2008).

CHEN, X et al., “XBP1 promotes triple-negative breast cancer by controlling the HIF 1 alpha pathway,” Nature, 508(7494): 103-107 (2014).

COFFMAN, R L et al., “Vaccine adjuvants: putting innate immunity to work,” Immunity, 33(4):492-503 (2010).

COUSSENS, L M et al., “Neutralizing tumor-promoting chronic inflammation: a magic bullet?” Science, 339(6117):286-291 (2013).

CRAPARO, E F and BONDI, M L, “Application of polymeric nanoparticles in immunotherapy,” Curr. Opin. Allergy Clin. Immunol., 12(6):658-664 (2012).

DAVE, B et al., “Targeting RPL39 and MLF2 reduces tumor initiation and metastasis in breast cancer by inhibiting nitric oxide synthase signaling,” Proc. Nat'l. Acad. Sci. USA, 111(24):8838-8843 (2014).

DINARELLO, C A, Interleukin 1 and interleukin 18 as mediators of inflammation and the aging process,” Amer. J. Clin. Nutr., 83(2):447S-455S (2006).

DOUGAN, M, and DRANOFF, G, “Immune therapy for cancer,” Annu. Rev. Immunol., 27:83-117 (2009).

DUGGAN, J M et al., “Synergistic interactions of TLR2/6 and TLR9 induce a high level of resistance to lung infection in mice,” J. Immunol., 186(10):5916-5926 (2011).

FACSDIVA Software V6.0, B. B., San Jose, Calif., USA (2007).

FRIC, J et al., “Phagocytosis of particulate antigens—all roads lead to calcineurin/NFAT signaling pathway,” Front. Immunol., 4:513 (2014).

FUERTES, M B et al., “Host type I IFN signals are required for antitumor CD8+ T cell responses through CD8{alpha}+dendritic cells,” J. Exp. Med., 208(10):2005-2016 (2011).

GAJEWSKI, T F et al., “Innate and adaptive immune cells in the tumor microenvironment,” Nat. Immunol., 14:1014-1022 (2013a).

GAJEWSKI, T F et al., “Cancer immunotherapy strategies based on overcoming barriers within the tumor microenvironment,” Curr. Opin. Immunol., 25(2):268-276 (2013b).

HALE, W G, and MARGHAM, J P, “HARPER COLLINS DICTIONARY OF BIOLOGY,” HarperPerennial, New York (1991).

HARDMAN, J G, and LIMBIRD, L E, (Eds.), “GOODMAN AND GILMAN′S THE PHARMACOLOGICAL BASIS OF THERAPEUTICS” 10^{th} Edition, McGraw-Hill, New York (2001).

HODI, F S et al., “Improved survival with ipilimumab in patients with metastatic melanoma,” N. Engl. J. Med., 363(8):711-723 (2010).

HOLM, C K et al., “Virus-cell fusion as a trigger of innate immunity dependent on the adaptor STING,” Nat. Immunol., 13(8):737-743 (2012).

JAIME-RAMIREZ, A C et al., “IL-12 enhances the antitumor actions of trastuzumab via NK cell IFN-gamma production,” J. Immunol., 186(6):3401-3409 (2011).

KAWAI, T, and AKIRA, S, “Toll-like receptors and their crosstalk with other innate receptors in infection and immunity,” Immunity, 34(5):637-650 (2011).

KLEINDIENST, P, and BROCKER, T, “Endogenous dendritic cells are required for amplification of T cell responses induced by dendritic cell vaccines in vivo,” J. Immunol., 170(6):2817-2823 (2003).

KOOL, M et al., “Alum adjuvant boosts adaptive mmunity by inducing uric acid and activating inflammatory dendritic cells,” J. Exp. Med., 205(4):869-882 (2008).LAN, K H et al., “A DNA vaccine against cytotoxic T-lymphocyte associated antigen-4 (CTLA-4) prevents tumor growth,” Biochem. Biophys. Res. Commun., 440(2):222-228 (2013).

L E BON, A, and TOUGH, D F, “Type I interferon as a stimulus for cross-priming,” Cytokine Growth Factor Rev., 19(1):33-40 (2008).

LUCCHINI, F et al., “Early and multifocal tumors in breast, salivary, harderian and epididymal tissues developed in MMTV-Neu transgenic mice,” Cancer Lett., 64(3):203-209 (1992).

MARABELLE, A et al., “Depleting tumor-specific Tregs at a single site eradicates disseminated tumors,” J. Clin. Invest., 123(6):2447-2463 (2013).

MERAZ, I M et al., “Multivalent presentation of MPL by porous silicon microparticles favors T helper 1 polarization enhancing the anti-tumor efficacy of doxorubicin nanoliposomes,” PloS One, 9(4):e94703 (2014).

MERAZ, I M et al., “Activation of the inflammasome and enhanced migration of microparticle-stimulated dendritic cells to the draining lymph node,” Mol. Pharm., 9(7):2049-2062 (2012).

MERAZ, I M et al., “Adjuvant cationic liposomes presenting MPL and IL-12 induce cell death, suppress tumor growth, and alter the cellular phenotype of tumors in a murine model of breast cancer,” Mol. Pharm., 11(10):3484-3491 (September 2014).

MILLIEN, V O et al., “Cleavage of fibrinogen by proteinases elicits allergic responses through Toll-like receptor 4,” Science, 341(6147):792-796 (2013).

MILLS, C D et al., “M-1/M-2 macrophages and the Th1/Th2 paradigm,” J. Immunol., 164(12):6166-6173 (2000).

MUKAI, Y et al., “Induction of endoplasmic reticulum-endosome fusion for antigen cross-presentation induced by poly (gamma-glutamic acid) nanoparticles,” J. Immunol., 187(12):6249-6255 (2011).

NAGATA, Y et al., “Peptides derived from a wild-type murine proto-oncogene c-erbB-2/HER2/neu can induce CTL and tumor suppression in syngeneic hosts,” J. Immunol., 159(3):1336-1343 (1997).

NAVA-PARADA, P et al., “Peptide vaccine given with a Toll-like receptor agonist is effective for the treatment and prevention of spontaneous breast tumors,” Cancer Res., 67(3):1326-1334 (2007).

NEEDLEMAN, S B and WUNSCH, C D, “A general method applicable to the search for similarities in the amino acid sequence of two proteins,” J. Mol. Biol., 48(3):443-453 (1970).

N G, D, and GOMMERMAN, J L, “The regulation of immune responses by DC-derived type I IFN,” Front. Immunol., 4:94 (2013).

O'SULLIVAN, T et al., “Cancer immunoediting by the innate immune system in the absence of adaptive immunity,” J. Exp. Med., 209(10):1869-1882 (2012).

PAULIS, L E et al., “Dendritic cell-based nanovaccines for cancer immunotherapy,” Curr. Opin. Immunol., 25(3):389-395 (2013).

PHAN, G Q and ROSENBERG, S A, “Adoptive cell transfer for patients with metastatic melanoma: the potential and promise of cancer immunotherapy,” Cancer Control, 20(4):289-297 (2013).

POSTOW, M A et al., “Immune checkpoint blockade in cancer therapy,” J. Clin. Oncol., doi: 10.1200/JCO.2014.59.4358 (2015).

PRINZ, M, and KNOBELOCH, K P, “Type I interferons as ambiguous modulators of chronic inflammation in the central nervous system,” Front. Immunol., 3:67 (2012).

SAUER, J D et al., “The N-ethyl-N-nitrosourea-induced Goldenticket mouse mutant reveals an essential function of Sting in the in vivo interferon response to Listeria monocytogenes and cyclic dinucleotides,” Infect. Immun., 79(2):688-694 (2011).

SHARMA, P et al., “Novel cancer immunotherapy agents with survival benefit: recent successes and next steps,” Nat. Rev. Cancer, 11(11):805-812 (2011).

SHARP, FA et al., “Uptake of particulate vaccine adjuvants by dendritic cells activates the NALP3 inflammasome,” Proc. Nat'l. Acad. Sci. USA, 106(3):870-875 (2009).

SHEN, H et al., “Enhancing chemotherapy response with sustained EphA2 silencing using multistage vector delivery,” Clin. Cancer Res., 19(7):1806-1815 (2013a).

SHEN, J et al., “High capacity nanoporous silicon carrier for systemic delivery of gene silencing therapeutics,” ACS Nano, 7(11):9867-9880 (2013b).

SINGLETON, P and SAINSBURY, D, “DICTIONARY OF MICROBIOLOGY AND MOLECULAR BIOLOGY,” 2^{nd} Ed., John Wiley and Sons, New York (1987).

SIMPSON-ABELSON, M R et al., “IL-12 delivered intratumorally by multilamellar liposomes reactivates memory T cells in human tumor microenvironments,” Clin. Immunol., 132(1):71-82 (2009).

SISTIGU, A et al., “Cancer cell-autonomous contribution of type I interferon signaling to the efficacy of chemotherapy,” Nat. Med., 20(11):1301-1309 (2014).

SPANER, D E et al., “Obstacles to effective Toll-like receptor agonist therapy for hematologic malignancies,” Oncogene, 27:208-217(2008).

SUMMERS DELUCA, L et al., “LTβR signaling in dendritic cells induces a type I IFN response that is required for optimal clonal expansion of CD8⁺ T cells,” Proc. Nat'l. Acad. Sci. USA, 108(5):2046-2051 (2011).

TANAKA, T et al., “Sustained small interfering RNA delivery by mesoporous silicon particles,” Cancer Res., 70(9):3687-3696 (2010).

TROMBETTA, E S, and MELLMAN, I, “Cell biology of antigen processing in vitro and in vivo,” Annu. Rev. Immunol., 23:975-1028 (2005).

WAN, S et al., “Chemotherapeutics and radiation stimulate MHC class I expression through elevated interferon-beta signaling in breast cancer cells,” PloS One, 7(3):e32542 (2012).

WAYTECK, L et al., “A personalized view on cancer immunotherapy,” Cancer Lett., 352(1):113-125 (2014).

X U, L G et al., “VISA is an adapter protein required for virus-triggered IFN-β signaling,” Mol. Cell, 19(6):727-740 (2005).

X U, R et al., “Multistage vectored siRNA targeting ataxia-telangiectasia mutated for breast cancer therapy,” Small, 9(9-10):1799-1808 (2013).

YANG, X et al., “Targeting the tumor microenvironment with interferon-β bridges innate and adaptive immune responses,” Cancer Cell, 25(1):37-48 (2014).

YEWDALL, A W et al., “CD8⁺ T cell priming by dendritic cell vaccines requires antigen transfer to endogenous antigen presenting cells,” PLoS One 5(6):e11144, (2010).

ZHANG, Z et al., “DDX1, DDX21, and DHX36 helicases form a complex with the adaptor molecule TRIF to sense dsRNA in dendritic cells,” Immunity, 34(6):866-878 (2011).

It should be understood that the examples and embodiments described herein are for illustrative purposes only and that various modifications or changes in light thereof will be suggested to persons skilled in the art and are to be included within the spirit and purview of this application and the scope of the appended claims.

All references, including publications, patent applications and patents, cited herein are hereby incorporated by reference to the same extent as if each reference was individually and specifically indicated to be incorporated by reference and was set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including,” or “containing,” with reference to an element or elements is intended to provide support for a similar aspect or embodiment of the invention that “consists of,” “consists essentially of,” or “substantially comprises,” that particular element or elements, unless otherwise stated or clearly contradicted by context (e.g., a composition described herein as comprising a particular element should be understood as also describing a composition that contains and/or that includes that particular element, unless otherwise explicated stated, or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically- and/or physiologically-related may be substituted for the agents described herein while the same or similar results would be achieved. All such similar substitutes and modifications apparent to those skilled in the art are deemed to be within the spirit, scope, and concept of the invention as defined by the appended claims.

What is claimed is:

1. A method of providing long-term protection against disease or cancer recurrence in a mammal, comprising administering to a mammalian subject in need thereof, an effective amount of a composition comprising a population of porous silicon (pSi) microparticles to provide long-term protection against the disease recurrence or the cancer recurrence in the mammal.

2. A method of providing long-term protection against disease or cancer recurrence in a mammal, comprising administering to a mammalian subject in need thereof, an effective amount of a composition comprising:
(a) a population of porous silicon (pSi) microparticles; and
(b) at least one tumor-specific, or cancer-specific antigen
to provide long-term protection against the disease recurrence or the cancer recurrence in the mammal.

3. The method of claim 1 or claim 2, wherein the cancer is diagnosed as, or is identified as, a refractory, a metastatic, a relapsed, or a treatment-resistant cancer.

4. The method of claim 1 or claim 2, wherein the disease is selected from the group consisting of West Nile Viral disease, acne, Alzheimer's disease, asthma, atherosclerosis, autoimmune disease, autoinflammatory disease, celiac disease, colitis, Crohn's disease, diabetes, glomerulonephritis, inflammatory bowel disease, irritable bowel syndrome, ischemia, Lupus, pelvic inflammatory disease, rheumatoid arthritis, and sarcoidosis.

5. The method of claim 1 or claim 2, wherein the composition is administered systemically to the mammal, either as a single injection, or as a series of multiple injections over a period of several days, several weeks, or several months or longer.

6. The method of claim 1 or claim 2, wherein administration of the composition increases expression of IFN-α4, IFN-β, or a combination thereof in one or more cells of the mammal.

7. The method of claim 1 or claim 2, wherein administration of the composition increases vaccination efficiency, stimulates antibody production, boosts memory T-cell populations, or any combination thereof in one or more cells of the mammal.

8. The method of claim 1 or claim 2, further comprising administration of an additional chemotherapeutic agent, or a chemoeffective amount of radiation.

9. The method of claim 1 or claim 2, wherein protection against the disease or the cancer recurrence persists from several weeks to several months following cessation of treatment.

10. The method of claim 1 or claim 2, wherein protection against the disease or the cancer recurrence persists from several months to several years following cessation of treatment.

11. The method of claim 1 or claim 2, wherein administration of the composition to suitable mammalian cells increases IFN-I expression or activates CD8- or CD4-positive cytotoxic T cells.

12. The method of claim 1 or claim 2, wherein administration of the composition to suitable mammalian cells depletes Treg cells, T-helper (T_{H}) cells, or a combination thereof.

13. The method of claim 1 or claim 2, wherein the composition further comprises a population of mammalian dendritic cells.

14. The method of claim 1 or claim 2, wherein the composition further comprises an adjuvant.

15. The method of claim 1 or claim 2, wherein the composition: (1) further comprises a population of liposomes, nanoparticles, or microparticles; or (2) is admixed with one or more surfactants, niosomes, ethosomes, transferosomes, phospholipids, or sphingosomes.

16. The method of claim 1 or claim 2, wherein the mammal is human.

17. The method of claim 2, wherein the antigen is specific for a tumor-specific peptide or polypeptide.

18. The method of claim 17, wherein the antigen is specific for an ovalbumin, HER2, or p66 peptide or polypeptide.

19. The method of claim 8, wherein the additional chemotherapeutic agent is selected from the group consisting of an immunomodulating agent, a neuroactive agent, an anti-inflammatory agent, an anti-lipidemic agent, a hormone, a receptor agonist, a receptor antagonist, an anti-infective agent, a protein, a peptide an antibody, an antigen-binding fragment, an enzyme, an RNA, a DNA, an siRNA, an mRNA, a ribozyme, a hormone, a cofactor, a steroid, an antisense molecule, and combinations thereof.

20. The method of claim 8, wherein the additional chemotherapeutic agent is selected from the group consisting of cyclophosphamide, doxorubicin, 5-fluorouracil, docetaxel, paclitaxel, trastuzumab, methotrexate, epirubicin, cisplatin, carboplatin, vinorelbine, capecitabine, gemcitabine, mitoxantrone, isabepilone, eribulin, lapatinib, carmustine, a nitrogen mustard, a sulfur mustard, a platin tetranitrate, vinblastine, etoposide, camptothecin, and combinations thereof.


SEQUENCE LISTING (2 sequences)

SEQ ID NO: 1 - protein, 8 aa, Artificial Sequence
  OVA-derived MHC class I epitope Synthetic Peptide
SIINFEKL

SEQ ID NO: 2 - protein, 17 aa, Artificial Sequence
  MHC class II epitope Synthetic Peptide
ISQAVHAAHAEINEAGR